(19) United States
(12) Patent Application Publication
(10) Pub. No.: US 2022/0000379 A1
(43) Pub. Date: Jan. 6, 2022

(54) CUFF UNIT, METHOD FOR MANUFACTURING CUFF UNIT, AND BLOOD PRESSURE MEASURING DEVICE

(71) Applicants: OMRON HEALTHCARE Co., Ltd., Kyoto (JP); OMRON Corporation, Kyoto (JP)

(72) Inventors: Masaki HARADA, Kyoto (JP); Takayuki MATSUOKA, Kyoto (JP); Minoru TANIGUCHI, Kyoto (JP)

(21) Appl. No.: 17/447,351

(22) Filed: Sep. 10, 2021

Related U.S. Application Data
(63) Continuation of application No. PCT/JP2020/005959, filed on Feb. 17, 2020.

(30) Foreign Application Priority Data
Mar. 15, 2019 (JP) 2019-048855

(51) Int. Cl. A61B 5/021; A61B 5/00

(52) U.S. Cl. CPC A61B 5/02141; A61B 2560/04; A61B 5/681

(57) ABSTRACT

A cuff unit, a method for manufacturing the cuff unit, and a blood pressure measuring device that allow suppressing a positional deviation in each of a pressing cuff and a sensing cuff with respect to a curler are provided. A cuff unit includes a pressing cuff joined to a curler and including a plurality of air bags that are layered and each inflated by a fluid, and a sensing cuff that includes one air bag inflated by a fluid and a joining margin formed on the air bag and joined to the air bag 81 adjacent to the air bag.

### CROSS-REFERENCE TO RELATED APPLICATIONS

This application is the U.S. national stage application filed pursuant to 35 U.S.C. 365(c) and 120 as a continuation of International Patent Application No. PCT/JP2020/005959, filed Feb. 17, 2020, which application claims priority to Japanese Patent Application No. 2019-048855, filed Mar. 15, 2019, which applications are incorporated herein by reference in their entireties.

### TECHNICAL FIELD

The present invention relates to a cuff unit used in a blood pressure measuring device, a method for manufacturing the cuff unit, and a blood pressure measuring device.

### BACKGROUND ART

In recent years, blood pressure measuring devices for measuring blood pressure are being used to monitor health status at home, as well as in medical facilities. A blood pressure measuring device detects vibration of the artery wall to measure blood pressure by, for example, inflating and contracting a cuff wrapped around the upper arm or the wrist of a living body and detecting the pressure of the cuff using a pressure sensor. As an example of the cuff used in such a blood pressure measuring device, the technology disclosed in JP 11-309119 A in which a plurality of air bags are inflated to compress an artery is known (for example, see Patent Document 1).

There is a demand for blood pressure measuring devices having an aspect of being attached to a wrist in which a sensing cuff is inflated in a direction in which blood vessels are occluded and the sensing cuff comes into close contact with a wrist when the sensing cuff is inflated. A technology that uses a curler between a belt, which fixes the blood pressure measuring device to the wrist, and a sensing cuff to bring the inflated sensing cuff into close contact with the wrist is known.

### CITATION LIST

### Patent Literature

Patent Document 1: JP 11-309119 A

### SUMMARY OF INVENTION

### Technical Problem

To bring the sensing cuff into close contact with the wrist, a blood pressure measuring device having a configuration in which a pressing cuff that presses the sensing cuff to the wrist is fixed to an inner circumferential surface of a curler is considered.

However, in the blood pressure measuring device having the configuration in which the pressing cuff and the sensing cuff are layered and disposed on the curler, when the pressing cuff and the sensing cuff are joined in order to the curler, positional deviation occurs in each of the pressing cuff and the sensing cuff with respect to the curler. The positional deviation in each of the pressing cuff and the sensing cuff increases a possibility of positional deviation of the sensing cuff with respect to the curler in some cases. The increase in the positional deviation of the sensing cuff with respect to the curler possibly decreases accuracy of blood pressure measurement.

Therefore, an object of the present invention is to provide a cuff unit, a method for manufacturing the cuff unit, and a blood pressure measuring device that allow suppressing a positional deviation in each of a pressing cuff and a sensing cuff with respect to a curler.

### Solution to Problem

According to one aspect, there is provided a cuff unit that includes a pressing cuff and a sensing cuff. The pressing cuff includes a plurality of first bag-like structures. The plurality of first bag-like structures are layered and each inflated by a fluid. The pressing cuff is joined to a curler. The sensing cuff includes one second bag-like structure and a joining margin. The one second bag-like structure is inflated by a fluid. The joining margin is formed on the second bag-like structure and joined to the first bag-like structure adjacent to the second bag-like structure.

Here, the cuff is wrapped around, for example, a wrist of a living body to measure a blood pressure and includes a single or multi-layer bag-like structures that are inflated by being supplied with a fluid. The bag-like structure is inflated by fluid, and is an air bag in a case where the fluid is air.

According to this aspect, configuring the cuff unit integrated by joining the pressing cuff and the sensing cuff with the joining margin allows the pressing cuff and the sensing cuff to be fixed to the curler as an integrated body. Thus, since the pressing cuff and the sensing cuff can be fixed in a single attachment work to the curler, positional displacement of the pressing cuff and the sensing cuff with respect to the curler can be suppressed compared with a configuration in which the pressing cuff and the sensing cuff are separately fixed to the curler. Furthermore, it is possible to suppress inhibiting the inflation of the pressing cuff by the joining margin.

In the cuff unit according to one aspect, there is provided a cuff unit as follows. the first bag-like structure is configured in a shape long in one direction. The second bag-like structure is configured in a shape long in one direction. The joining margin is formed at an outer edge portion along a longitudinal direction of the second bag-like structure. The joining margin is joined to the outer edge portion along the longitudinal direction of the first bag-like structure.

According to this aspect, the joining margin is joined to a wide range of the first bag-like structure. As a result, this allows suppressing an entrance of water, such as sweat of a user, between the pressing cuff and the sensing cuff.

In the cuff unit according to one aspect, there is provided a cuff unit as follows. The pressing cuff and the sensing cuff are curved following an inner circumferential surface of the curler.

According to this aspect, a wrinkle, which inhibits the inflation, can be suppressed in the pressing cuff and the sensing cuff when the cuff unit is joined to the curler.

In the cuff unit according to one aspect, there is provided a cuff unit as follows. The cuff unit includes a back plate disposed between the pressing cuff and the sensing cuff and joined to the pressing cuff and the sensing cuff.

According to this aspect, the integrated body of the pressing cuff, the back plate, and the sensing cuff can be fixed to the curler in a single attachment operation, and therefore the number of manufacturing steps of the blood pressure measuring device can be reduced.

According to one aspect, there is provided a method for manufacturing a cuff unit that includes: disposing a first structure including a pressing cuff on a placement surface of a jig to position the first structure with respect to the placement surface formed in a curved surface corresponding to a surface of a curler to which the pressing cuff is joined; disposing a second structure including a sensing cuff on the first structure to position the second structure with respect to the placement surface; and joining and integrating the first structure and the second structure.

Here, the placement surface formed as the curved surface corresponding to the surface of the curler on which the pressing cuff is fixed is a curved surface that, when the cuff unit formed along this placement surface is fixed to the curler, the cuff unit curves more than that before the cuff unit is fixed to the curler, and this allows suppressing wrinkles, which inhibit the inflation, in at least one of the pressing cuff and the sensing cuff. The curved surface of the curler corresponding to the surface on which the pressing cuff is disposed is, for example, a curved surface having a curvature same as that of the surface of the curler on which the pressing cuff is disposed. In another example, the curved surface of the curler corresponding to the surface on which the pressing cuff is disposed is a curved surface having a curvature substantially the same as that of the surface of the curler on which the pressing cuff is disposed.

According to this aspect, by integrally fixing the pressing cuff and the sensing cuff using the jig, which has the placement surface configured to have the curved surface corresponding to the surface of the curler to which the pressing cuff is fixed, the pressing cuff and the sensing cuff can be integrally fixed in a curved posture. As a result, when the cuff unit in which the pressing cuff and the sensing cuff are integrally fixed is fixed to the curler, a state in which a wrinkle occurs in the pressing cuff and the sensing cuff and the inflation of the pressing cuff and the sensing cuff is inhibited can be suppressed. That is, when the pressing cuff and the sensing cuff are integrally fixed using a jig having a planar placement surface to configure a cuff unit, the cuff unit is configured in which a top surface of the sensing cuff has a planar shape according to the planar placement surface. When this cuff unit is fixed to the curler, a difference between an inner circumference and an outer circumference generated by curving the cuff unit according to the shape of the curler possibly generates wrinkles in the pressing cuff and the sensing cuff.

However, using the jig having the curved placement surface, the pressing cuff and the sensing cuff are fixed to be integrated. This allows suppressing wrinkles, which inhibit the inflation, in the pressing cuff and the sensing cuff when the cuff unit is fixed to the curler.

In the method for manufacturing the cuff unit according to one aspect, there is provided a method for manufacturing a cuff unit as follows. The second structure includes a joining margin. The first structure and the second structure are joined at the joining margin.

According to this aspect, another component is not required for joining the first structure and the second structure. This makes it possible to suppress an increase in the number of components of the integrated body of the first structure and the second structure.

In the method for manufacturing the cuff unit according to one aspect, there is provided a method for manufacturing a cuff unit that includes, after disposing a back plate on the first structure and joining the back plate to the first structure, disposing the second structure on the first structure and the back plate to join the second structure to the first structure and the back plate.

According to this aspect, the integrated body of the pressing cuff, the back plate, and the sensing cuff can be fixed to the curler in a single attachment operation, and therefore the number of manufacturing steps of the blood pressure measuring device can be reduced.

In the method for manufacturing the cuff unit according to one aspect, there is provided a method for manufacturing a cuff unit as follows. The jig includes a plurality of positioning pins. The first structure includes a plurality of holes in which the positioning pins are disposed. The second structure includes a plurality of holes in which the positioning pins are disposed. The joining margin is joined to the first structure with the plurality of positioning pins set to the plurality of holes in the first structure and the plurality of holes in the second structure.

According to this aspect, movement of the first structure and the second structure relative to the placement surface, such as rotation around the positioning pins, can be suppressed.

In the method for manufacturing the cuff unit according to one aspect, there is provided a method for manufacturing a cuff unit as follows. The first structure and the second structure each include a cutting margin. After the first structure and the second structure are fixed and integrated, the cutting margins are cut.

According to this aspect, after fixing and integrating the first structure and the second structure, the cutting is performed, thereby ensuring improvement of work efficiency of manufacturing the cuff unit.

According to one aspect, there is provided a blood pressure measuring device that includes a curler, a cuff unit, and a device body. The cuff unit includes a pressing cuff and a sensing cuff. The pressing cuff includes a plurality of first bag-like structures. The plurality of first bag-like structures are layered and each inflated by a fluid. The pressing cuff is joined to a curler. The sensing cuff includes one second bag-like structure and a joining margin. The one second bag-like structure is inflated by a fluid. The joining margin is formed on the second bag-like structure and joined to the first bag-like structure adjacent to the second bag-like structure. The device body supplies the fluid to the cuff unit.

According to this aspect, configuring the cuff unit integrated by joining the pressing cuff and the sensing cuff with the joining margin allows the pressing cuff and the sensing cuff to be fixed to the curler as the integrated body. Thus, since the pressing cuff and the sensing cuff can be fixed in a single attachment work to the curler, positional displacement of the pressing cuff and the sensing cuff with respect to the curler can be suppressed compared with a configuration in which the pressing cuff and the sensing cuff are separately fixed to the curler. Furthermore, it is possible to suppress inhibiting the inflation of the pressing cuff by the joining margin.

### Advantageous Effects of Invention

The present invention allows providing the cuff unit, the method for manufacturing the cuff unit, and the blood pressure measuring device that allow suppressing the positional deviation of each of the pressing cuff and the sensing cuff with respect to the curler.

### BRIEF DESCRIPTION OF DRAWINGS

FIG. 1 is a perspective view illustrating the configuration of a blood pressure measuring device according to an embodiment of the present invention.

FIG. 2 is an exploded perspective view illustrating the configuration of the blood pressure measuring device.

FIG. 3 is a side view illustrating the configuration of the blood pressure measuring device.

FIG. 4 is an explanatory diagram illustrating a state in which the blood pressure measuring device is attached to the wrist.

FIG. 5 is a block diagram illustrating the configuration of the blood pressure measuring device.

FIG. 6 is a perspective view illustrating the configuration of the blood pressure measuring device.

FIG. 7 is an exploded perspective view illustrating the configuration of a curler and a cuff structure of the blood pressure measuring device.

FIG. 8 is a cross-sectional view illustrating the configurations of the curler and a cuff unit of the blood pressure measuring device.

FIG. 9 is a cross-sectional view illustrating the configurations of the curler and the cuff unit of the blood pressure measuring device.

FIG. 10 is a cross-sectional view illustrating the configuration of a tensile cuff of the blood pressure measuring device.

FIG. 11 is a cross-sectional view illustrating the configuration of the tensile cuff of the blood pressure measuring device.

FIG. 12 is a perspective view illustrating the configuration of the curler of the blood pressure measuring device.

FIG. 13 is a plan view illustrating a configuration of a cuff structure of the blood pressure measuring device.

FIG. 14 is a plan view illustrating the configuration of the cuff structure.

FIG. 15 is a plan view illustrating a configuration of a pressing cuff of the blood pressure measuring device.

FIG. 16 is a cross-sectional view illustrating the configuration of the pressing cuff.

FIG. 17 is a plan view illustrating the configuration of a sensing cuff of the blood pressure measuring device.

FIG. 18 is a cross-sectional view illustrating the configuration of the sensing cuff.

FIG. 19 is a plan view illustrating the configuration of the cuff unit of the blood pressure measuring device.

FIG. 20 is a plan view illustrating the configuration of the tensile cuff of the blood pressure measuring device.

FIG. 21 is a cross-sectional view illustrating the configuration of the tensile cuff of the blood pressure measuring device.

FIG. 22 is a flowchart depicting an example of a method for manufacturing a first structure.

FIG. 23 is a plan view illustrating a configuration of a first sheet.

FIG. 24 is a plan view illustrating a configuration of a second sheet.

FIG. 25 is a plan view illustrating a configuration of a third sheet.

FIG. 26 is a plan view illustrating a configuration of a fourth sheet.

FIG. 27 is an explanatory diagram illustrating an example of one process of the method for manufacturing the first structure.

FIG. 28 is an explanatory diagram illustrating an example of one process of the method for manufacturing the first structure.

FIG. 29 is an explanatory diagram illustrating an example of one process of the method for manufacturing the first structure.

FIG. 30 is an explanatory diagram illustrating an example of one process of the method for manufacturing the first structure.

FIG. 31 is an explanatory diagram illustrating an example of one process of the method for manufacturing the first structure.

FIG. 32 is an explanatory diagram illustrating an example of one process of the method for manufacturing the first structure.

FIG. 33 is an explanatory diagram illustrating an example of one process of the method for manufacturing the first structure.

FIG. 34 is a flowchart depicting an example of a method for manufacturing the second structure.

FIG. 35 is a plan view illustrating a configuration of a fifth sheet.

FIG. 36 is a plan view illustrating a configuration of a sixth sheet.

FIG. 37 is an explanatory diagram illustrating an example of one process of the method for manufacturing the second structure.

FIG. 38 is an explanatory diagram illustrating an example of one process of the method for manufacturing the second structure.

FIG. 39 is a flowchart depicting an example of a method for manufacturing the cuff unit.

FIG. 40 is a perspective view illustrating a configuration of a jig.

FIG. 41 is an explanatory diagram illustrating an example of one process of the method for manufacturing the cuff unit.

FIG. 42 is an explanatory diagram illustrating an example of one process of the method for manufacturing the cuff unit.

FIG. 43 is an explanatory diagram illustrating an example of one process of the method for manufacturing the cuff unit.

FIG. 44 is a flowchart depicting an example of a method for manufacturing the tensile cuff.

FIG. 45 is a flowchart depicting an example of a method for manufacturing the blood pressure measuring device.

FIG. 46 is a flowchart depicting an example of usage of the blood pressure measuring device.

FIG. 47 is a perspective view illustrating an example in which the blood pressure measuring device is attached to a wrist.

FIG. 48 is a perspective view illustrating an example in which the blood pressure measuring device is attached to the wrist.

FIG. 49 is a perspective view illustrating an example in which the blood pressure measuring device is attached to the wrist.

FIG. 50 is a cross-sectional view schematically illustrating a state in which the blood pressure measuring device is attached to the wrist.

FIG. 51 is an explanatory diagram illustrating a modified example of one process of a method for manufacturing the cuff unit.

FIG. 52 is a cross-sectional view illustrating a modified example of the cuff unit and a configuration of a curler.

FIG. 53 is a cross-sectional view illustrating another modified example of the cuff unit and a configuration of a curler.

### DESCRIPTION OF EMBODIMENTS

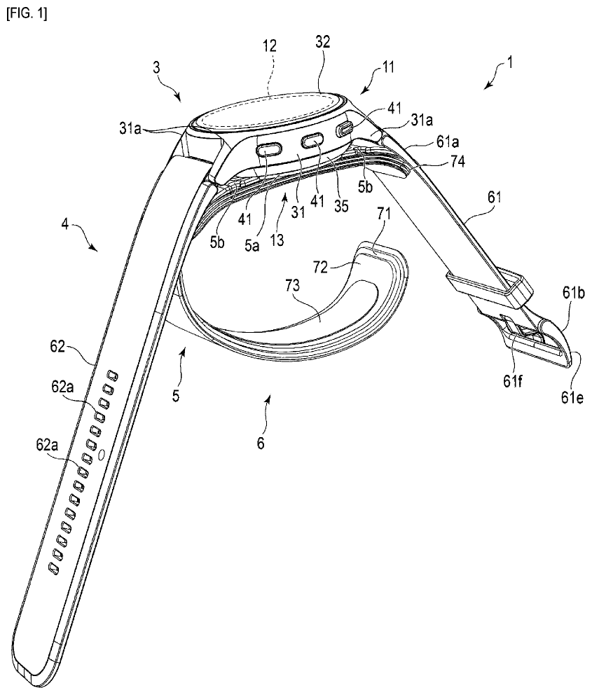

An example of a blood pressure measuring device 1 according to an embodiment of the present invention is described below using FIGS. 1 to 21.

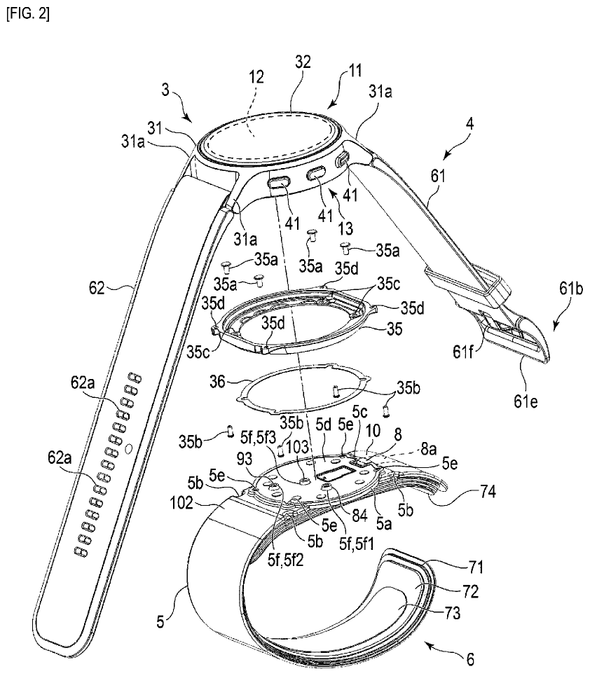

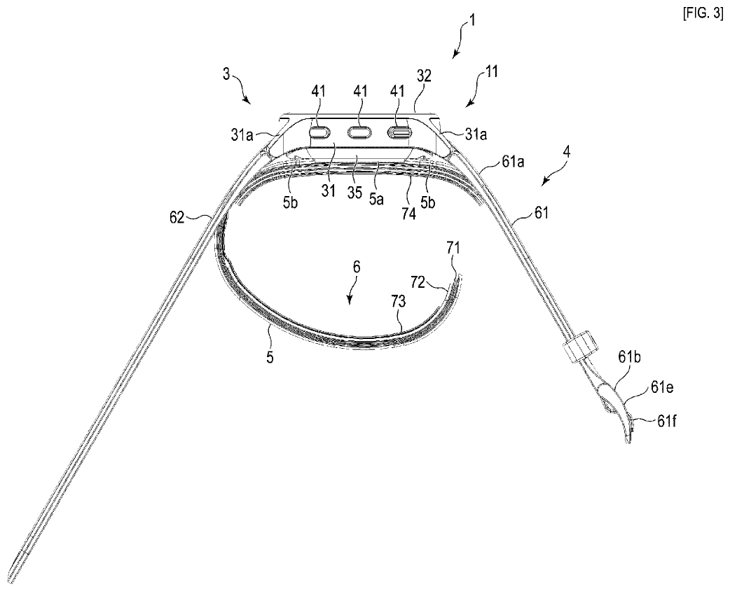

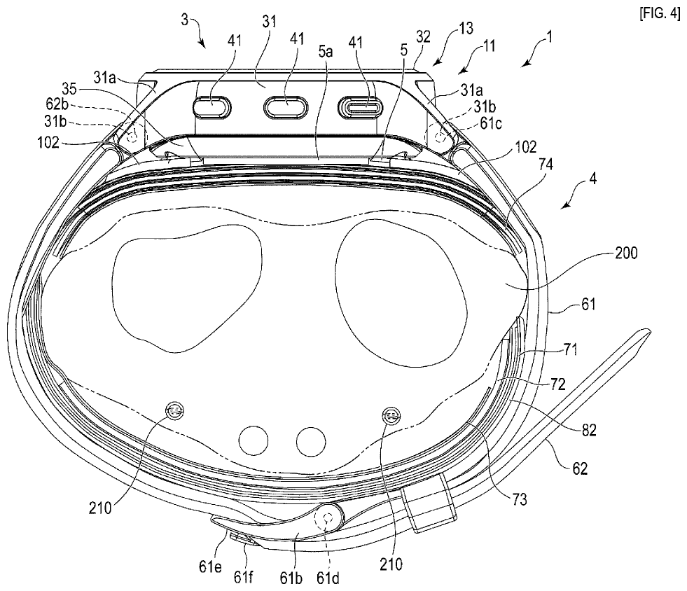

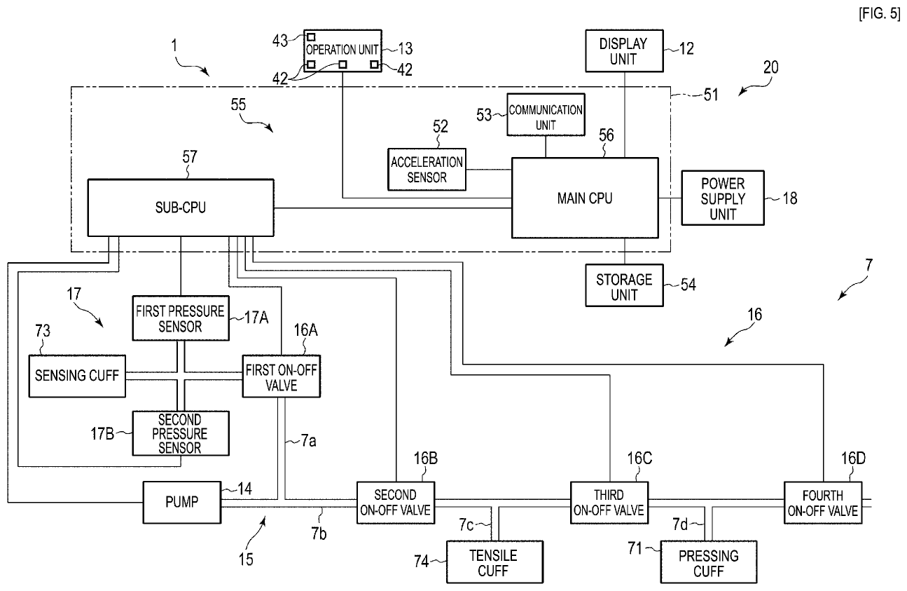

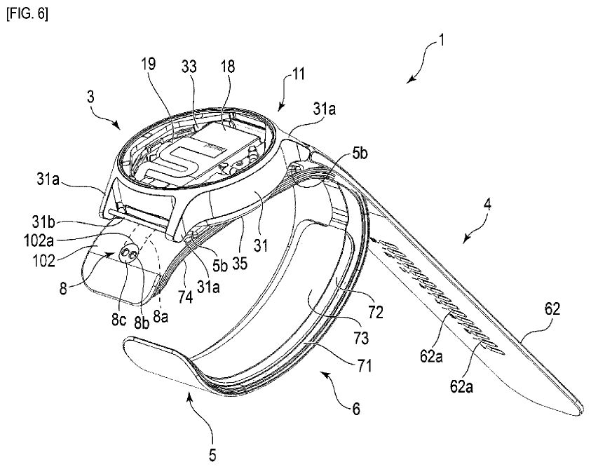

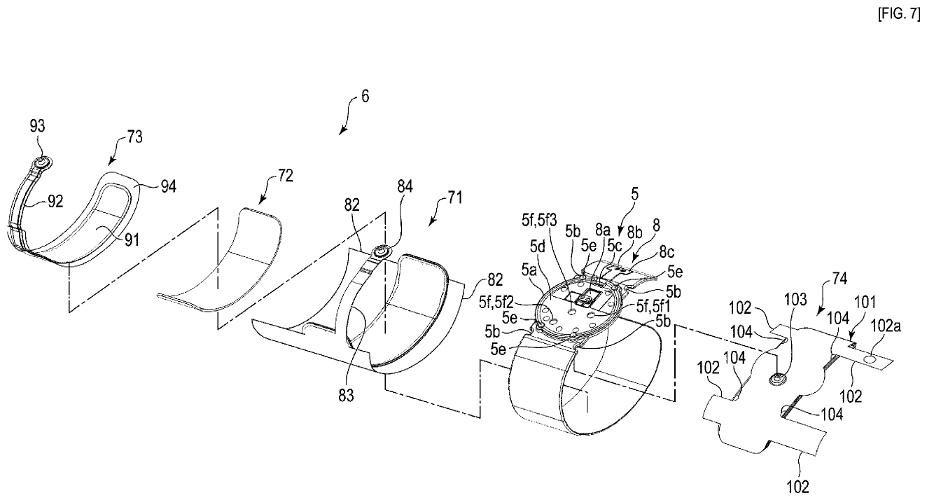

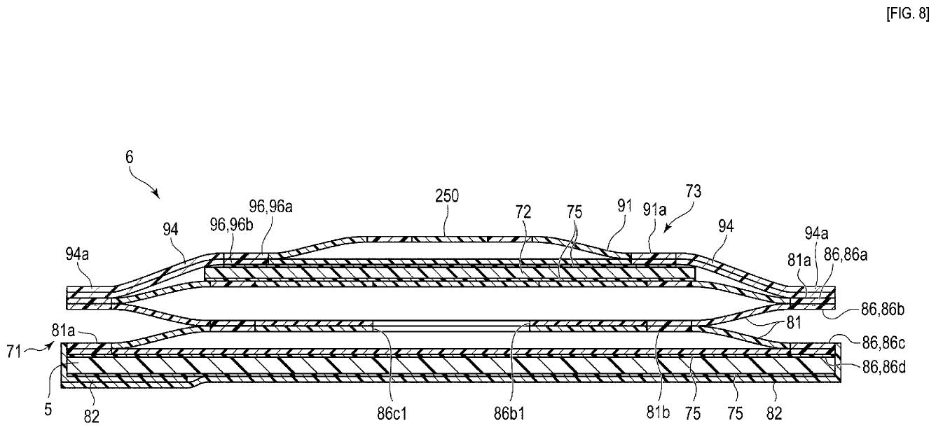

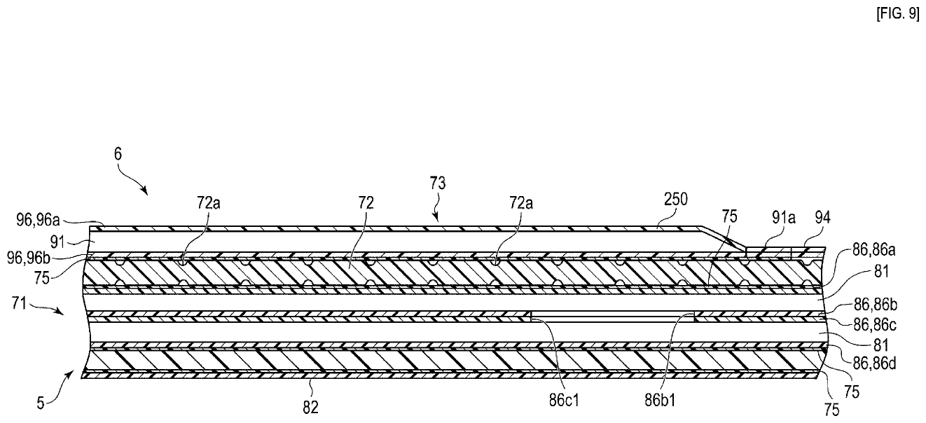

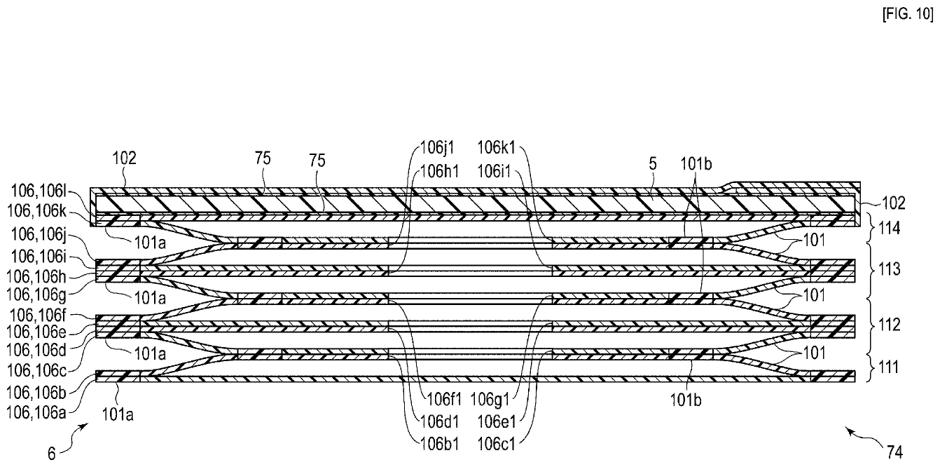

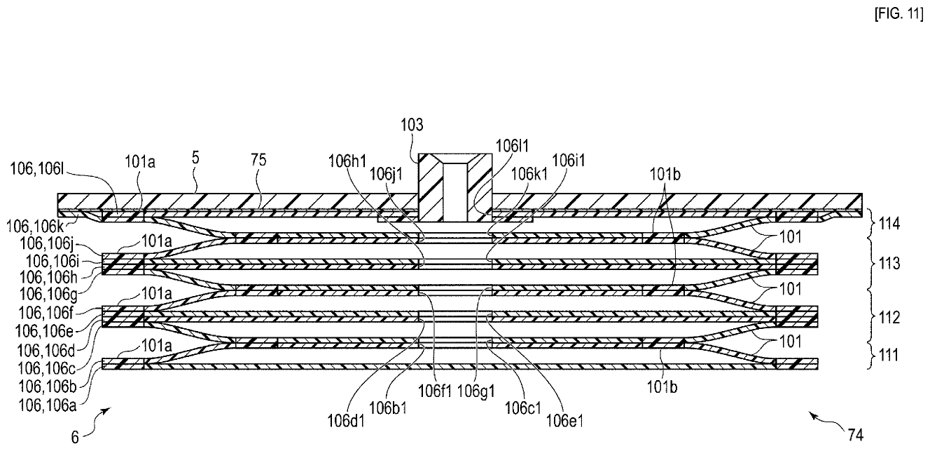

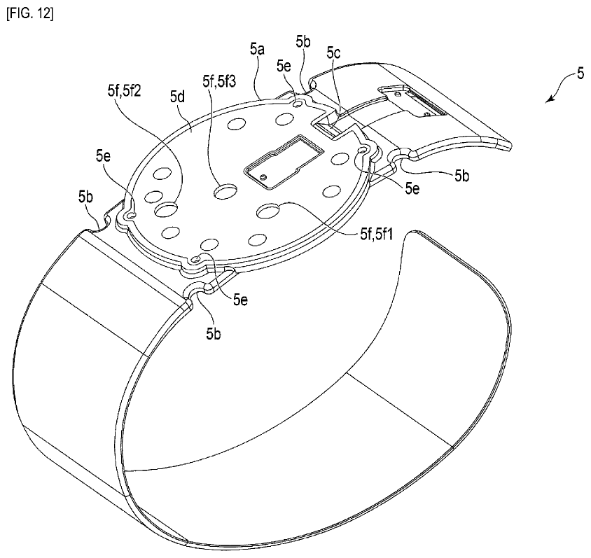

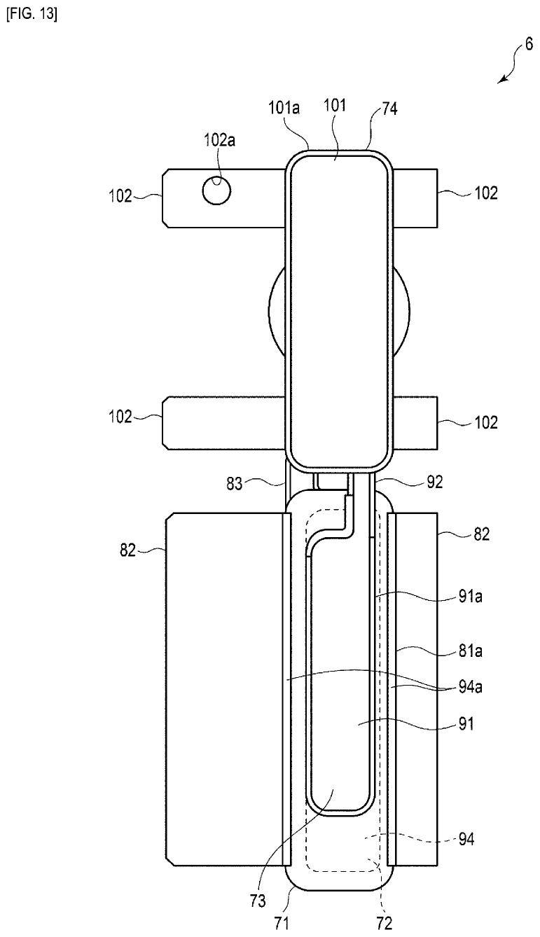

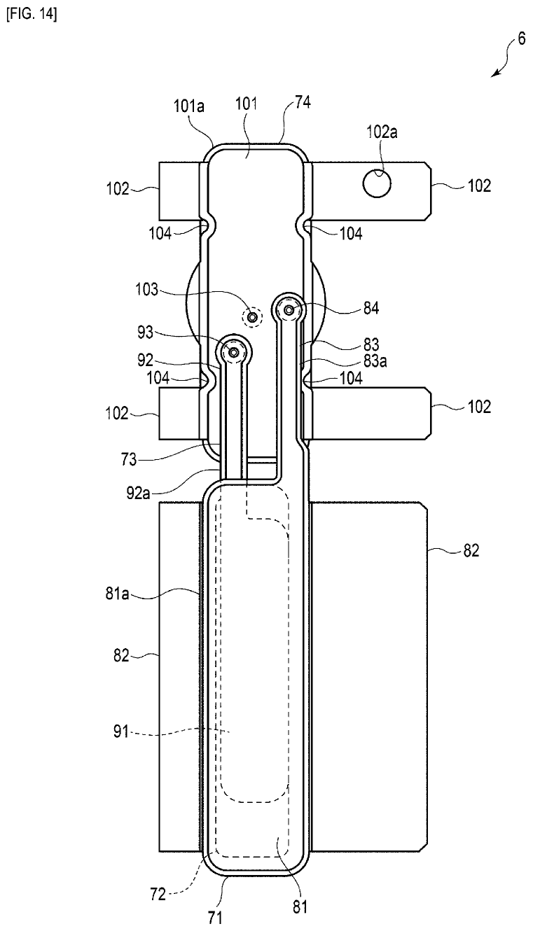

FIG. 1 is a perspective view illustrating a configuration of the blood pressure measuring device 1 according to the present embodiment. FIG. 2 is an exploded perspective view illustrating the configuration of the blood pressure measuring device 1. FIG. 3 is a side view illustrating the configuration of the blood pressure measuring device 1. FIG. 4 is an explanatory diagram illustrating a state in which the blood pressure measuring device 1 is attached to a wrist 200. FIG. 5 is a block diagram illustrating the configuration of the blood pressure measuring device 1. FIG. 6 is a perspective view illustrating the configuration of the blood pressure measuring device 1 with some configurations removed. FIG. 7 is an exploded perspective view illustrating the configuration of a curler 5 and a cuff structure 6 of the blood pressure measuring device 1. FIG. 8 is a cross-sectional view illustrating configurations of the curler 5 and a cuff unit 250 of the blood pressure measuring device 1. FIG. 9 is a cross-sectional view illustrating the configurations of the curler 5 and the cuff unit 250 of the blood pressure measuring device 1. FIG. 10 is a cross-sectional view illustrating the configuration of a tensile cuff 74 of the blood pressure measuring device 1. FIG. 11 is a cross-sectional view illustrating the configuration of the tensile cuff 74 of the blood pressure measuring device 1. FIG. 12 is a perspective view illustrating the configuration of the curler 5 of the blood pressure measuring device 1. FIG. 13 is a plan view illustrating a configuration of the cuff structure 6 of the blood pressure measuring device 1 from the wrist 200 side. FIG. 14 is a plan view illustrating the configuration of the curler 5 of the cuff structure 6 on the inner circumferential surface side.

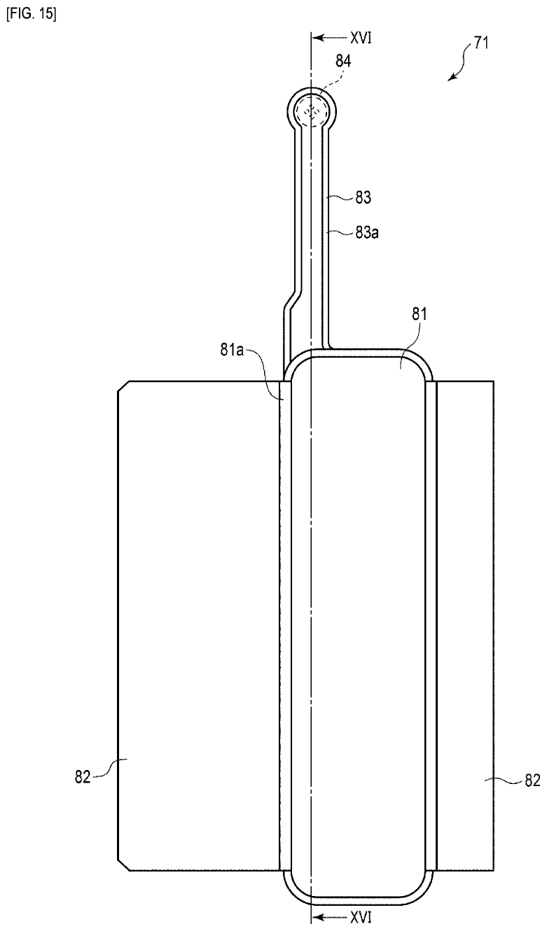

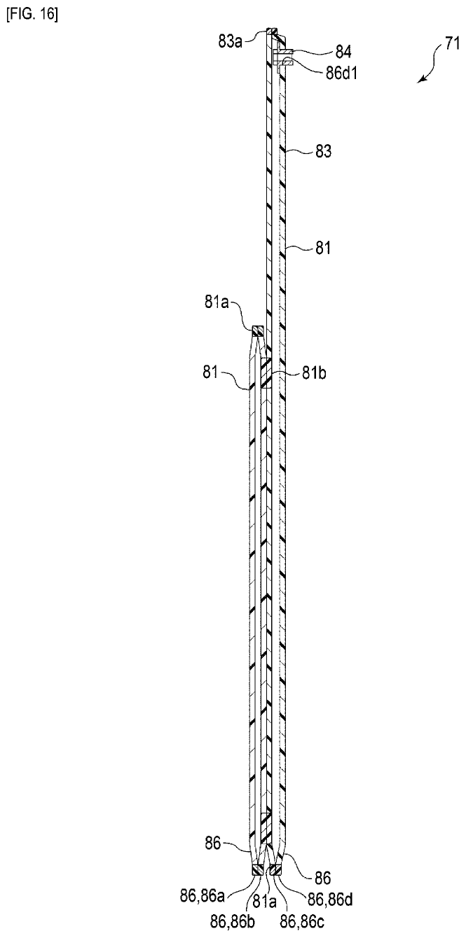

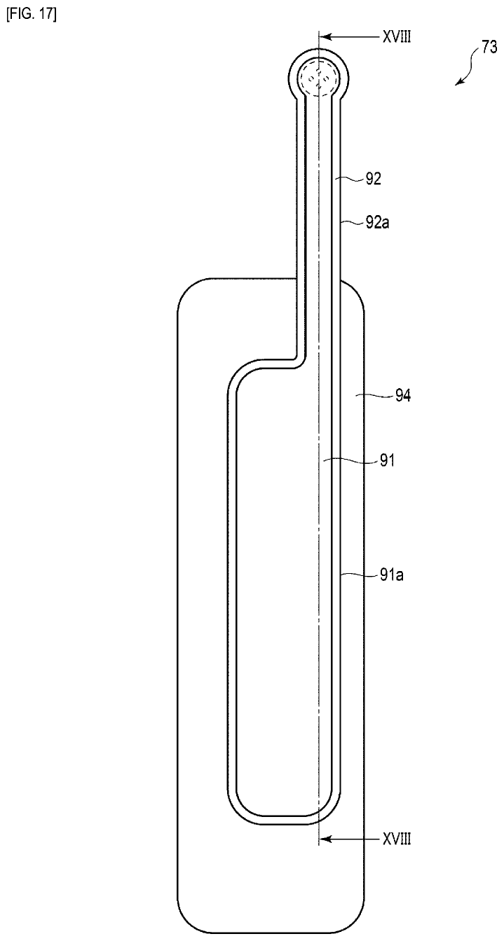

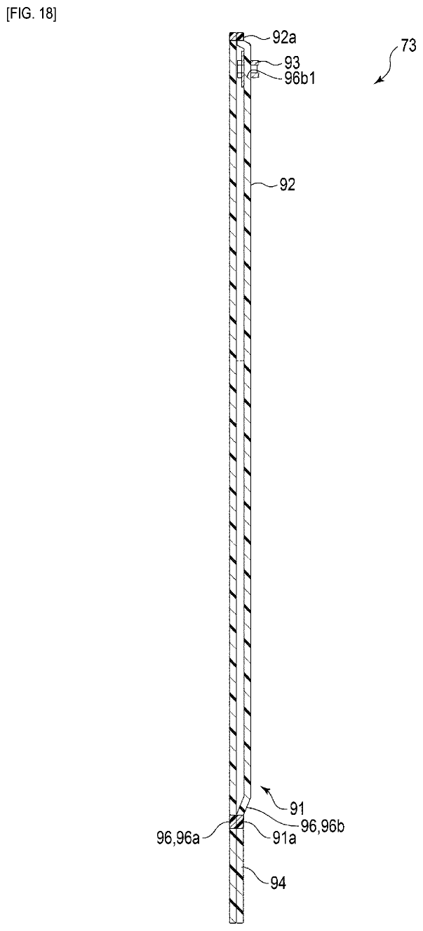

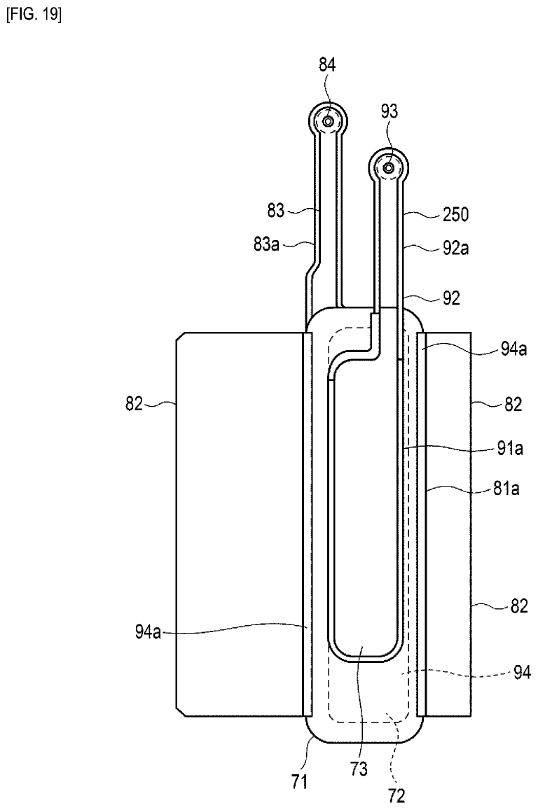

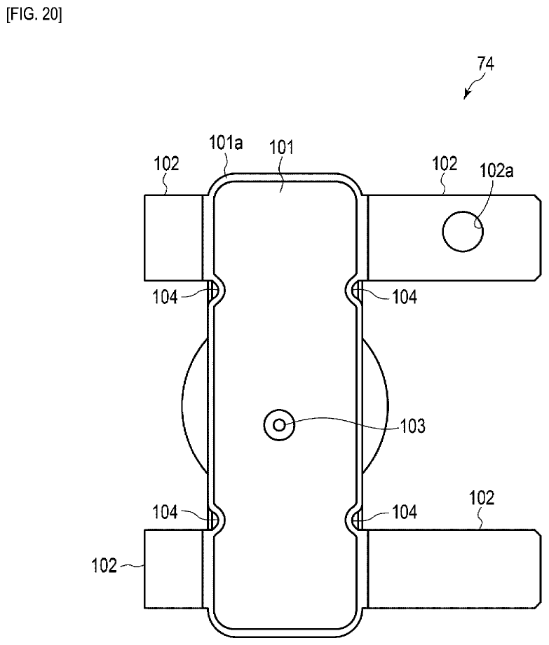

FIG. 15 is a plan view illustrating the configuration of a pressing cuff 71 of the blood pressure measuring device 1. FIG. 16 is a cross-sectional view illustrating the configuration of the pressing cuff 71, which is a line cross-section along XVI-XVI illustrated in FIG. 15. FIG. 17 is a plan view illustrating the configuration of a sensing cuff 73 of the blood pressure measuring device 1. FIG. 18 is a cross-sectional view illustrating the configuration of the sensing cuff 73 of the blood pressure measuring device 1, which is a line cross-section along XVIII-XVIII illustrated in FIG. 17. FIG. 19 is a plan view illustrating the configuration of the cuff unit 250 of the blood pressure measuring device 1. FIG. 20 is a plan view illustrating the configuration of the tensile cuff 74 of the blood pressure measuring device 1. FIG. 21 is a cross-sectional view illustrating the configuration of the tensile cuff 74.

The blood pressure measuring device 1 is an electronic blood pressure measuring device attached to a living body. The present embodiment will be described using an electronic blood pressure measuring device having an aspect of a wearable device attached to the wrist 200 of the living body.

As illustrated in FIGS. 1 to 6, the blood pressure measuring device 1 includes a device body 3, a belt 4 that fixes the device body 3 at the wrist, the curler 5 disposed between the belt 4 and the wrist, the cuff structure 6 including the pressing cuff 71, the sensing cuff 73, and the tensile cuff 74, a fluid circuit 7 fluidly connecting the device body 3 and the cuff structure 6, and a power feeding unit 8 provided on the curler 5.

As illustrated in FIGS. 1 to 6, the device body 3 includes, for example, a case 11, a display unit 12, an operation unit 13, a pump 14, a flow path portion 15, the on-off valve 16, the pressure sensor 17, a power supply unit 18, a vibration motor 19, and a control substrate 20. The device body 3 supplies a fluid to the cuff structure 6 using the pump 14, the on-off valve 16, the pressure sensor 17, the control substrate 20, and the like.

As illustrated in FIGS. 1 to 3, the case 11 includes a tubular outer case 31, a windshield 32 covering an opening of the outer case 31 on the opposite side (outer side) to the wrist 200 side, a base portion 33 provided inside the outer case 31 on the wrist 200 side, a rear cover 35 covering the wrist 200 side of the outer case 31, and a sealing member 36 provided on the lower surface of the rear cover 35.

The outer case 31 is formed in, for example, a cylindrical shape. The outer case 31 includes pairs of lugs 31a provided at respective symmetrical positions in the circumferential direction of an outer circumferential surface, and spring rods 31b each provided between each of the two pairs of lugs 31a. The windshield 32 is, for example, a circular glass plate.

The base portion 33 holds the display unit 12, the operation unit 13, the pump 14, the on-off valve 16, the pressure sensor 17, the power supply unit 18, the vibration motor 19, and the control substrate 20. Additionally, the base portion 33 constitutes a portion of the flow path portion 15 that makes the pump 14 and the cuff structure 6 fluidly continuous.

The rear cover 35 is constituted in an annular shape with an open center. The rear cover 35 covers the end portion on the outer peripheral edge side of the outer case 31 on the wrist 200 side. With the rear cover 35 configured as such being integrally assembled with the curler 5, the central opening is covered by the curler 5, and the rear cover 35 together with the curler 5 forms a rear lid covering the end portion of the outer case 31 on the wrist 200 side. Specifically, the rear cover 35 is fixed to the curler 5 with four first joining members 35a and fixed to the end portion of the outer case 31 on the wrist 200 side with four second joining members 35b. The rear cover 35 includes four hole portions 35c into which the first joining members 35a that are provided at the bottom portion and fixed to the curler 5 are inserted, and four hole portions 35d provided at four portions of the outer circumferential portion that radially project out, into which the second joining members 35b that are fixed to the outer case 31 are inserted.

The first joining members 35a and the second joining members 35b are members, such as a screw, a bolt, a machine screw, a rive, for mechanically joining two components. In the present embodiment, the first joining members 35a and the second joining members 35b are screws.

The sealing member 36 is a double-sided tape, for example, formed in the shape of the region of the rear cover 35 that comes into contact with the curler 5. The sealing member 36 seals between the curler 5 and the rear cover 35 by being provided between the curler 5 and the rear cover 35.

The display unit 12 is disposed on the base portion 33 of the outer case 31 and directly below the windshield 32. As illustrated in FIG. 5, the display unit 12 is electrically connected to the control substrate 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, the operation unit 13 includes a plurality of buttons 41 provided on the case 11, a sensor 42 that detects operation of the buttons 41, and a touch panel 43 provided on the display unit 12 or the windshield 32, as illustrated in FIGS. 1 and 5. When operated by the user, the operation unit 13 converts an instruction into an electrical signal. The sensor 42 and the touch panel 43 are electrically connected to the control substrate 20 to output electrical signals to the control substrate 20.

As the plurality of buttons 41, for example, three buttons are provided. The buttons 41 are supported by the base portion 33 and protrude from the outer circumferential surface of the outer case 31. The plurality of buttons 41 and a plurality of the sensors 42 are supported by the base portion 33. The touch panel 43 is integrally provided on the windshield 32, for example.

The pump 14 is, for example, a piezoelectric pump. The pump 14 compresses air and supplies compressed air to the cuff structure 6 through the flow path portion 15. The pump 14 is electrically connected to the control substrate 20.

The flow path portion 15 constitutes the flow path connecting from the pump 14 to the pressing cuff 71 and the tensile cuff 74 and a flow path connecting from the pump 14 to the sensing cuff 73, as illustrated in FIG. 5. Additionally, the flow path portion 15 constitutes a flow path connecting from the pressing cuff 71 and the tensile cuff 74 to the atmosphere, and a flow path connecting from the sensing cuff 73 to the atmosphere. The flow path portion 15 is a flow path of air constituted by a hollow portion, a groove, a flow path tank, a tube, or the like provided in the base portion 33 and the like.

The on-off valve 16 opens and closes a portion of the flow path portion 15. Specifically, a plurality of on-off valves 16, specifically four on-off valves 16 are provided, for example, as illustrated in FIG. 5, and selectively open and close the flow path connecting from the pump 14 to the pressing cuff 71 and the tensile cuff 74, the flow path connecting from the pump 14 to the sensing cuff 73, the flow path connecting from the pressing cuff 71 and the tensile cuff 74 to the atmosphere, and the flow path connecting from the sensing cuff 73 to the atmosphere, by the combination of opening and closing of each of the on-off valves 16. As a specific example, the four on-off valves 16 are constituted by a first on-off valve 16A, a second on-off valve 16B, a third on-off valve 16C, and a fourth on-off valve 16D. The first on-off valve 16A opens and closes the flow path connecting the pump 14 and the sensing cuff 73. The second on-off valve 16B opens and closes the flow path connecting the pump 14 and the tensile cuff 74. The second on-off valve 16B and the third on-off valve 16C open and close the flow path connecting the pump 14 and the pressing cuff 71. The second on-off valve 16B, the third on-off valve 16C, and the fourth on-off valve 16D open and close the flow path connecting the pump 14 and the atmosphere.

A pressure sensor 17 at least detects the pressure of the sensing cuff 73. The pressure sensor 17 is provided with a first pressure sensor 17A and a second pressure sensor 17B, for example. The pressure sensor 17 converts a detected pressure into an electrical signal, and outputs the electrical signal to the control substrate 20. For example, the first pressure sensor 17A and the second pressure sensor 17B are provided in the flow path connecting the first on-off valve 16A of the flow path portion 15 and the sensing cuff 73. These flow paths are continuous through the pressing cuff 71, the sensing cuff 73, and the tensile cuff 74 to the pump 14 by the opening and closing of each of the on-off valves, and thus the pressure in these flow paths corresponds to the pressure in the internal space of the pressing cuff 71, the sensing cuff 73, and the tensile cuff 74 connecting to the pump 14.

Specifically, for example, the pressure sensor 17 detects the pressure of the sensing cuff 73, i.e., the pressure of the flow path portion 15 connecting the pump 14 and the sensing cuff 73, when the first on-off valve 16A is open and the second on-off valve 16B is closed. Also, the pressure sensor 17 detects the pressure of the sensing cuff 73 and the tensile cuff 74, i.e., the pressure of the flow path portion 15 connecting the pump 14, the sensing cuff 73, and the tensile cuff 74, when the first on-off valve 16A and the second on-off valve 16B are open and the third on-off valve 16C is closed. Furthermore, the pressure sensor 17 detects the pressure of the pressing cuff 71, the sensing cuff 73, and the tensile cuff 74, i.e., the pressure of the flow path portion 15 connecting the pump 14, the pressing cuff 71, the sensing cuff 73, and the tensile cuff 74, when the first on-off valve 16A, the second on-off valve 16B, and the third on-off valve 16C are open and the fourth on-off valve 16D is open or closed.

The power supply unit 18 is, for example, a secondary battery such as a lithium ion battery. The power supply unit 18 is electrically connected to the control substrate 20, as illustrated in FIG. 5. The power supply unit 18 supplies power to the control substrate 20.

As illustrated in FIG. 5, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base portion 33 of the case 11 using screws or the like.

The acceleration sensor 52 is, for example, a 3-axis acceleration sensor. The acceleration sensor 52 outputs, to the control unit 55, an acceleration signal representing acceleration of the device body 3 in three directions orthogonal to one another. For example, the acceleration sensor 52 is used to measure, from the detected acceleration, the amount of activity of a living body to which the blood pressure measuring device 1 is attached.

The communication unit 53 is configured to be capable to transmit and receive information to and from an external device wirelessly or by wire. For example, the communication unit 53 transmits information controlled by the control unit 55, and information of a measured blood pressure value, a pulse, and the like to an external device via a network, and receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, or the like.

The storage unit 54 pre-stores program data for controlling the overall blood pressure measuring device 1 and the fluid circuit 7, settings data for setting various functions of the blood pressure measuring device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measuring device 1 and operation of the fluid circuit 7. The control unit 55 is electrically connected to and supplies power to the display unit 12, the operation unit 13, the pump 14, each of the on-off valves 16 and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 5, the control unit 55 includes a main Central Processing Unit (CPU) 56 that controls operation of the overall blood pressure measuring device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensor 17, and outputs an image signal corresponding to the measurement results to the display unit 12.

For example, the sub-CPU 57 drives the pump 14 and the on-off valves 16 to feed compressed air to the pressing cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signal output by the pressure sensors 17. The sub-CPU 57 controls the pump 14 and the on-off valves 16 to selectively feed compressed air to the pressing cuff 71 and the sensing cuff 73 and selectively depressurize the pressing cuff 71 and the sensing cuff 73.

As illustrated in FIGS. 1 to 4, the belt 4 includes a first belt 61 provided on the first pair of lugs 31a and a first spring rod 31b, and a second belt 62 provided on the second pair of lugs 31a and a second spring rod 31b. The belt 4 is wrapped around the wrist 200 with the curler 5 in between.

The first belt 61 is referred to as a so-called a parent and is configured like a band capable of being joined to the second belt 62. As illustrated in FIGS. 1 to 3, the first belt 61 includes a belt portion 61a and a buckle 61b. The belt portion 61a is configured like a band. The belt portion 61a is formed of an elastically deformable resin material. In addition, the belt portion 61a is flexible and includes a sheet-like insert member inside the belt portion 61a for suppressing stretching in the longitudinal direction of the belt portion 61a. The belt portion 61a includes a first hole portion 61c that is formed at one end portion and extends orthogonal to the longitudinal direction of the belt portion 61a, and a second hole portion 61d that is formed at the other end portion and extends orthogonal to the longitudinal direction of the first belt 61.

As illustrated in FIGS. 4 and 6, the first hole portion 61c is provided at the end portion of the belt portion 61a The first hole portion 61c has an inner diameter at which the spring rod 31b can be inserted into the first hole portion 61c and at which the first belt 61 can rotate with respect to the spring rod 31b. In other words, the first belt 61 is rotatably held by the outer case 31 by disposing the first hole portion 61c between the pair of lugs 31a and around the spring rod 31b.

As illustrated in FIGS. 1 and 3, the second hole portion 61d is provided at the leading end of the belt portion 61a The buckle 61b is attached to the second hole portion 61d.

As illustrated in FIGS. 1 and 3, the buckle 61b includes a frame body 61e in a rectangular frame shape and a prong 61f rotatably attached to the frame body 61e. A side of the frame body 61e to which the prong 61f is attached is inserted into the second hole portion 61d, and the frame body 61e is mounted rotatably with respect to the belt portion 61a.

The second belt 62 is referred to as a so-called blade tip, and is configured in a band-like shape having a width at which the second belt 62 can be inserted into the frame body 61e. The second belt 62 is formed of an elastically deformable resin material. In addition, the second belt 62 is flexible and includes a sheet-like insert member inside the second belt 62 for suppressing stretching in the longitudinal direction of the second belt 62.

In addition, as illustrated in FIGS. 1 and 2, the second belt 62 includes a plurality of small holes 62a into which the prong 61f is inserted. Additionally, the second belt 62 includes a third hole portion 62b provided at first end portion of the second belt 62 and extending orthogonally to the longitudinal direction of the second belt 62. The third hole portion 62b has an inner diameter at which the spring rod 31b can be inserted into the third hole portion 62b and at which the second belt 62 can rotate with respect to the spring rod 31b. In other words, the second belt 62 is rotatably held by the outer case 31 by disposing the third hole portion 62b between the pair of lugs 31a and around the spring rod 31b.

The second belt 62 is inserted into the frame body 61e, and the prong 61f is inserted into the small hole 62a, and thus the first belt 61 and the second belt 62 are integrally connected together, and the belt 4 as described above, together with the outer case 31, comes to have an annular shape following along the circumferential direction of the wrist 200. By shaping the belt 4 in an annular shape following along the circumferential direction of the wrist 200, the curler 5 is pressed and elastically deformed to follow along the circumferential direction of the wrist of the wearer of the blood pressure measuring device 1.

As illustrated in FIGS. 1 to 4, the curler 5 is configured in a band-like shape that curves in such a manner as to follow along the circumferential direction of the wrist 200. The curler 5 is formed in a shape in which a first end and a second end spaced apart from each other. For example, a first end side outer surface of the curler 5 is fixed to the rear cover 35 of the device body 3. The curler 5 is disposed at a position where the first end and the second end protrude more to one side of the wrist 200 than the rear cover 35. Accordingly, the curler 5 is disposed with the first end and the second end to one side of the wrist 200 when the blood pressure measuring device 1 is attached to the wrist 200. Furthermore, the first end and the second end of the curler 5 are located adjacent to each other at a predetermined distance from each other. The curler 5 is formed of a resin material, for example. In a specific example, the curler 5 is formed of a polypropylene with a thickness of approximately 1 mm.

In a specific example, as illustrated in FIGS. 1 to 4, the curler 5 is configured in a band-like shape that curves following along the circumferential direction of the wrist. Furthermore, the curler 5 includes a disk-like cover portion 5a provided at a position facing the hand back side of the wrist 200 on the first end side, and constitutes the rear lid together with the rear cover 35, and an escape portion 5b that is provided in the peripheral region of the cover portion 5a and allows the second joining members 35b that fix the outer case 31 and the rear cover 35 to be moveable. For example, the cover portion 5a and the adjacent portion of the cover portion 5a of the curler 5 are formed in a plate-like shape, and the first and second end sides is formed curving with a predetermined curvature more than the cover portion 5a. Furthermore, the length of the curler 5 from the cover portion 5a to the first end is less than the length from the cover portion 5a to the second end. In a specific example, the shorter side of the curler 5 from the cover portion 5a to the first end is disposed on the hand back side of the wrist, and the longer side from the cover portion 5a to the second end extends from the hand back side of the wrist, passing through one side, to the hand palm-side of the wrist 200.

Additionally, as illustrated in FIG. 12, the curler 5 is formed in a shape with the second end located at the inner circumferential surface side of the first end side when the first end and the second end are brought close. In a specific example, the width of the curler 5 in the width direction of the wrist 200 is set to be greater on the hand back side of the wrist 200 than on the hand palm-side of the wrist 200. Furthermore, the radius of curvature of the first end of the curler 5 on the hand back side of the wrist 200 is set to be greater than the radius of curvature of the second end on the hand palm-side of the wrist 200. According to such a configuration, when both end sides of the curler 5 are brought to abut, the second end is disposed further to the inward side of the curler 5 than the first end. Furthermore, the curler 5 is provided with a recess 5c provided adjacent to the cover portion 5a on a portion of the cover portion 5a, on the outer surface on the first end side from the cover portion 5a, and also on the outer surface on the shorter side extending from the cover portion 5a.

The cover portion 5a includes an insert member 5d for reinforcement which is inserted. The cover portion 5a is fixed to the wrist 200 side of the outer case 31 with the fixed rear cover 35 in between. The cover portion 5a includes screw holes 5e provided at positions facing the four hole portions 35c of the rear cover 35, into which the first joining members 35a for fixing the rear cover 35 are screwed. Further, the cover portion 5a includes three hole portions 5f for connecting the cuff structure 6 to the device body 3.

The escape portion 5b is an escape portion for disposing the second joining members 35b in the rear cover 35 and for disposing a tool for rotating the second joining members 35b in a manner so that the second joining members 35b do not interfere with the curler 5 when the rear cover 35 is fixed to the outer case 31 from the rear cover 35 side with the second joining members 35b.

The three hole portions 5f include a first hole portion 5f1 formed with an inner diameter into which a connection portion 84 described below of the pressing cuff 71 can be inserted, a second hole portion 5f2 formed with an inner diameter into which a connection portion 93 described below of the sensing cuff 73 can be inserted, and a third hole portion 5f3 formed with an inner diameter into which a connection portion 103 described below of the tensile cuff 74 can be inserted. In the present embodiment, the second hole portion 5f2 is disposed in the cover portion 5a closer to the second end side on the hand palm-side of the curler 5 than the first hole portion 5f1 and the third hole portion 5f3.

The curler 5 with such a configuration is fixed to the outer case 31 with the first end and the second end orientated to face the second belt 62 of the belt 4. Also, the curler 5 at least at the position facing the hand palm-side of the wrist 200 curves along the circumferential direction on the hand palm-side of the wrist 200, and thus the cuff structure 6 facing the hand palm-side of the wrist 200 is held in a curved state following along the shape of the hand palm-side of the wrist 200.

The curler 5 has a hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist.

The cuff structure 6 is disposed on an inner circumferential surface of the curler 5, and is held along the shape of the inner circumferential surface of the curler 5. As a specific example, the cuff structure 6 is held by disposing the pressing cuff 71 and the tensile cuff 74 on the inner circumferential surface of the curler 5, and fixing the cuff structure 6 by a joining layer 75 provided between the curler 5 and the pressing cuff 71 and the tensile cuff 74. In the present embodiment, the joining layer 75 is adhesive or double-sided tape.

As illustrated in FIGS. 1 to 6, 13, and 14, the cuff structure 6 includes the pressing cuff 71, a back plate 72, the sensing cuff 73, and the tensile cuff 74. Also, the cuff structure 6 is provided with the joining layer 75 for joining components each other and joining the curler 5 and the cuffs 71 and 74. The cuff structure 6 is fixed to the curler 5. The cuff structure 6 includes the pressing cuff 71, the back plate 72, and the sensing cuff 73 that are stacked one another and disposed on the curler 5, and the tensile cuff 74 that is spaced apart from the pressing cuff 71, the back plate 72, and the sensing cuff 73 and disposed on the curler 5.

In a specific example, as illustrated in FIG. 4, the cuff structure 6 is fixed to the inner circumferential surface of the curler 5 on the hand palm-side of the wrist 200 with the pressing cuff 71, the back plate 72, and the sensing cuff 73 stacked in this order from the inner circumferential surface of the curler 5 toward the wrist 200 side. In addition, the cuff structure 6 includes the tensile cuff 74 disposed on the inner circumferential surface of the curler 5 on the hand back side of the wrist 200. Each of the members of the cuff structure 6 is fixed to an adjacent member of the cuff structure 6 in a stacking direction by the joining layer 75.

The pressing cuff 71 is fluidly connected to the pump 14 through the flow path portion 15. The pressing cuff 71 is inflated to pressing the back plate 72 and the sensing cuff 73 toward the wrist 200 side. As illustrated in FIGS. 8, 9, and 13 to 17, the pressing cuff 71 includes a plurality of, for example, two-layer air bags (first bag-like structures) 81, a target join portion 82 provided on the air bag 81 facing the curler 5, a flow path body 83 communicating with the air bag 81, and the connection portion 84 provided on the leading end of the flow path body 83. The pressing cuff 71 with such a configuration is configured by integrally welding a plurality of sheet members 86 together.

Here, the air bags 81 are bag-like structures, and in the present embodiment, the blood pressure measuring device 1 is configured to use air with the pump 14, and thus the present embodiment will be described using the air bags. However, in a case where a fluid other than air is used, the bag-like structures may be fluid bags that are inflated by a fluid. The plurality of air bags 81 are stacked and are in fluid communication with one another in the stacking direction.

Each of the air bags 81 is formed in a rectangular bag-like shape that is long in one direction. Additionally, the air bags 81 are set so that the width in the lateral direction is the same as the width in the lateral direction of the curler 5. The air bag 81 is constituted by, for example, combining two sheet members 86 and, as illustrated in FIGS. 8, 9, and 13 to 17, welding weld portions 81a using heat into a rectangular frame shape long in one direction. In addition, the two-layer air bags 81 are formed by integrally combining the two air bags 81 by welding using heat, or welding the facing sheet members 86 of the adjacent air bags 81 and after that welding the air bags 81. In a specific example, the two-layer air bags 81 are fluidly continuous through openings provided in the sheet members 86 facing one another. In addition, in the two-layer air bags 81, by bridge welding the opposing sheet members 86 together in a quadrilateral frame shape smaller than the weld portion 81a located on the outer peripheral edge and surrounding the plurality of openings with a bridge weld portion 81b, the adjacent air bags 81 are integrally formed and made to be fluidly continuous on the inner side of the bridge weld portion 81b. Here, “bridge” in bridge welding and the bridge weld portion means integrally joining adjacent air bags 81.

A single or a plurality of target join portions 82 are provided at at least a portion of the edge portion of the air bag 81 disposed adjacent to the curler 5. The target join portion 82 is formed by a portion of the sheet member 86 forming the air bag 81.

An example of the present embodiment will be described using the examples illustrated in FIGS. 7 to 9 and 13 to 15 in which one target join portion 82 is provided on the edge portion in the lateral direction of each of the air bags 81. Note that, for example, the target join portion 82 may be divided in the longitudinal direction of the air bag 81 by a slit, or a plurality of target join portions 82 may be provided in the longitudinal direction of the air bag 81. The target join portion 82 is at least joined to the outer circumferential surface of the curler 5 when the pressing cuff 71 is disposed on the inner circumferential surface of the curler 5. Furthermore, for example, two target join portions 82 are stacked and welded.

Note that the two target join portions 82 are set to have a different length to the length in the lateral direction of the air bags 81, for example. In this example, the two target join portions 82 are stacked and welded at the first end side in the lateral direction of the curler 5. Note that as long as the two target join portions 82 are able to be disposed with the leading end on the outer circumferential surface of the curler 5, the length is able to be set as appropriate, and the two target join portions 82 may be stackable or not. However, in a case where the length is set to a stackable length, the length is preferably a length such that the leading end does not extend further out than the outer edge of the outer circumferential surface of the curler 5.

As illustrated in FIGS. 7 and 13 to 17, the flow path body 83 is integrally provided on a single air bag 81, for example, on a portion of the edge portion at the first end in the longitudinal direction of the air bag 81 adjacent to the curler 5. As a specific example, the flow path body 83 is provided at the end portion of the air bag 81 near the device body 3. Additionally, the flow path body 83 is formed in a shape that is long in one direction and has less width than the width of the air bag 81 in the lateral direction and formed with a leading end having a circular shape. The flow path body 83 includes the connection portion 84 on the leading end. The flow path body 83 is connected to the flow path portion 15 through the connection portion 84 and constitutes a flow path between the flow path portion 15 of the device body 3 and the air bag 81.

The flow path body 83 is constituted by welding a portion of sheet members 86, which is adjacent to a region of the sheet members 86 constituting the air bags 81, in a frame shape long in one direction using heat, in a state where the connection portion 84 is disposed on the two sheet members 86. The flow path body 83 with such a configuration is disposed between the inner circumferential surface of the curler 5 and the tensile cuff 74, and the leading end is disposed at a position facing the first hole portion 5f1 on the main surface on the wrist 200 side of the region where the cover portion 5a of the curler 5 is provided.

Note that, a portion of the weld portion 81a, where the two sheet members 86 are welded in a rectangular frame shape, is not welded and the air bags 81 provided with the flow path body 83 are constituted to be continuous with a weld portion 83a constituting the flow path body 83, and thus the air bags 81 are fluidly continuous with the flow path body 83.

The connection portion 84 is, for example, a nipple. The connection portion 84 is provided at the leading end of the flow path body 83. The leading end of the connection portion 84 is exposed from the sheet member 86, facing the curler 5, of the two sheet members 86 constituting the flow path body 83. The connection portion 84 is inserted in the first hole portion 5f1 of the cover portion 5a and is connected to the flow path portion 15.

As a specific example, as illustrated in FIGS. 8, 9, and 52, the pressing cuff 71 having such a configuration includes a first sheet member 86a, a second sheet member 86b, a third sheet member 86c, and a fourth sheet member 86d from the wrist 200 side. The second sheet member 86b constitutes the first-layer air bag 81 along with the first sheet member 86a, the third sheet member 86c is integrally joined to the second sheet member 86b and constitutes the target join portion 82, and the fourth sheet member 86d constitutes the second-layer air bag 81 and the flow path body 83 along with the third sheet member 86c. Note that the pressing cuff 71 is integrally constituted by joining adjacent sheet members 86 by welding using heat.

The first sheet member 86a and the second sheet member 86b are configured in a similar rectangular shape to the air bags 81, and peripheral edge portions of the four sides are welded to constitute the air bags 81. The second sheet member 86b and the third sheet member 86c are disposed facing each other, and include a plurality of openings 86b1 and 86c1, respectively, through which the two air bags 81 are fluidly continuous. Additionally, the second sheet member 86b and the third sheet member 86c are integrally joined by the peripheral region of the plurality of openings 86b1 and 86c1 being bridge welded using heat in a quadrilateral frame shape smaller than the welded four sides of the air bags 81.

The third sheet member 86c, for example, is constituted in a shape that allows the air bags 81, the target join portion 82, and the flow path body 83 to be constituted. The fourth sheet member 86d, for example, is constituted in a shape that allows the air bags 81 and the flow path body 83 to be constituted. Furthermore, the fourth sheet member 86d includes a hole portion 86d1 into which the leading end of the connection portion 84 can be inserted, for example.

The air bags 81, the target join portion 82, and the flow path body 83 are constituted by the third sheet member 86c and the fourth sheet member 86d being disposed facing one another, welded using heat along the peripheral edge shape of the air bag 81 and the flow path body 83 so that the air bag 81 and the flow path body 83 are fluidly continuous, and cut in a predetermined shape.

The hole portion 86d1 of the fourth sheet member 86d is disposed with the connection portion 84, and the peripheral region of the hole portion 86d1 is welded to the connection portion 84 using heat. Furthermore, the fourth sheet member 86d is joined with the inner circumferential surface of the curler 5 with the joining layer 75 in between, and the target join portion 82 of the third sheet member 86c is joined to the outer circumferential surface of the curler 5 with the joining layer 75 in between.

As illustrated in FIGS. 8, 9 and 52, the back plate 72 is applied to the outer surface of the first sheet member 86a of the pressing cuff 71 by the joining layer 75. The back plate 72 is formed in a plate shape using a resin material. The back plate 72 is made of polypropylene, for example, and is formed into a plate shape having a thickness of approximately 1 mm. The back plate 72 has shape followability.

Here, “shape followability” refers to a function of the back plate 72 by which the back plate 72 can be deformed in such a manner as to follow the shape of a contacted portion of the wrist 200 to be disposed, the contacted portion of the wrist 200 refers to a region of the wrist 200 that is faced by the back plate 72. Here, the contact as used herein includes both direct contact and indirect contact with the sensing cuff 73 in between.

For example, as illustrated in FIG. 9, the back plate 72 includes a plurality of grooves 72a extending in both main surfaces of the back plate 72 in a direction orthogonal to the longitudinal direction. The plurality of grooves 72a face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 is deformed in such a manner as to follow to the shape of the wrist 200, and has shape followability of extending in the circumferential direction of the wrist. The back plate 72 is formed such that the length of the back plate 72 is sufficient to cover the hand palm-side of the wrist 200. The back plate 72 transfers the pressing force from the pressing cuff 71 to the back plate 72 side main surface of the sensing cuff 73 in a state in which the back plate 72 is extending along the shape of the wrist 200.

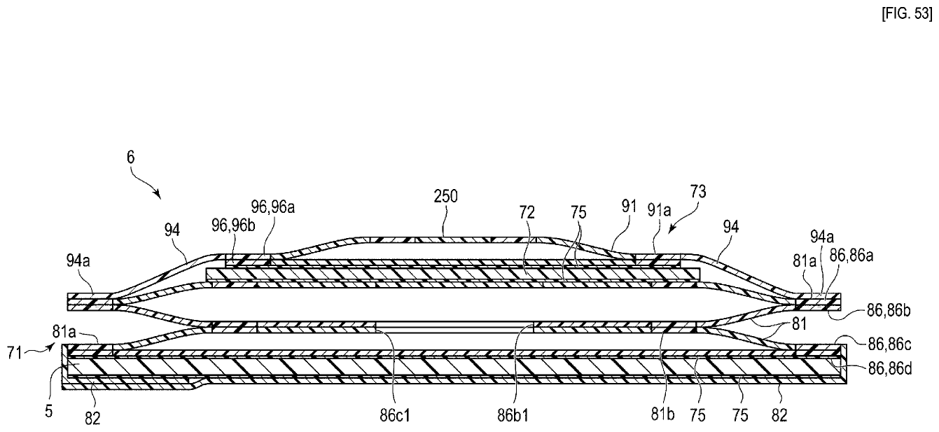

The sensing cuff 73 is fluidly connected to the pump 14 through the flow path portion 15. The sensing cuff 73 is fixed to the main surface of the back plate 72 on the wrist 200 side. For example, the sensing cuff 73 is in direct contact with a region of the wrist 200 where an artery 210 resides, as illustrated in FIG. 4 and FIG. 52. The artery 210 as used herein is the radial artery and the ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm-side region of the wrist 200 in which the artery 210 resides. The sensing cuff 73 is pressed by the inflated pressing cuff 71 toward the wrist 200 side with the back plate 72 in between.

As a specific example, as illustrated in FIGS. 8, 9, 13, 14, 17, and 18, the sensing cuff 73 includes one air bag (a second bag-like structure) 91, a flow path body 92 that communicates with the air bag 91, the connection portion 93 provided at the leading end in the flow path body 92, and a joining margin 94 at least a portion of which is joined to the pressing cuff 71. One main surface of the air bag 91 of the sensing cuff 73 is fixed to the back plate 72. For example, the sensing cuff 73 is joined to the main surface of the back plate 72 on the wrist 200 side by the joining layer 75. The sensing cuff 73 with such a configuration is constituted by welding two sheet members 96.

Here, the air bag 91 is a bag-like structure, and in the present embodiment, the blood pressure measuring device 1 is configured to use air with the pump 14, and thus the present embodiment will be described using the air bag. However, in a case where a fluid other than air is used, the bag-like structure may be a fluid bag and the like.

The air bag 91 is constituted in a rectangular shape that is long in one direction. The air bag 91 is constituted by, for example, combining two sheet members 96 and, as illustrated in FIGS. 8, 9, 13, 14, 17, and 18, welding weld portions 91a using heat into a rectangular frame shape long in one direction.

The flow path body 92 is integrally provided at a portion of the edge portion of the first end in the longitudinal direction of the air bag 91. As a specific example, the flow path body 92 is provided at the end portion of the air bag 91 near the device body 3. Additionally, the flow path body 92 is formed in a shape that is long in one direction and has less width than the width of the air bag 91 in the lateral direction, and formed with a leading end having a circular shape. The flow path body 92 includes the connection portion 93 on the leading end. The flow path body 92 includes the connection portion 93 on the leading end. The flow path body 92 is connected to the flow path portion 15 through the connection portion 93 and constitutes a flow path between the flow path portion 15 of the device body 3 and the air bag 91.

The flow path body 92 is constituted by welding a portion of sheet members 96, which is adjacent to a region of the sheet members 96 constituting the air bag 91, in a frame shape long in one direction using heat, in a state where the connection portion 93 is disposed on the two sheet members 96. Note that, a portion of the weld portion 91a, where the two sheet members 96 are welded in a rectangular frame shape, is not welded and the air bag 91 is constituted to be continuous with a weld portion 92a constituting the flow path body 92, and thus the air bag 91 and the flow path body 92 are fluidly continuous. The flow path body 92 with such a configuration is disposed between the inner circumferential surface of the curler 5 and the tensile cuff 74, and the leading end is disposed at a position facing the second hole portion 5f2 on the main surface on the wrist 200 side of the region where the cover portion 5a of the curler 5 is provided.

The connection portion 93 is, for example, a nipple. The connection portion 93 is provided at the leading end of the flow path body 92. Also, the leading end of the connection portion 93 is externally exposed from the sheet member 96 facing the curler 5 and the back plate 72, of the two sheet members 96 constituting the flow path body 92. The connection portion 93 is inserted in the second hole portion 5f2 of the cover portion 5a and is connected to the flow path portion 15.

The joining margin 94 is formed to be joinable to the air bag 81 adjacent to the air bag 91 among the plurality of air bags 81 of the pressing cuff 71, in other words, a portion of the weld portion 81a of the air bag 81 on the back plate 72 side by welding. As a specific example, the joining margin 94 is joined to the outer edge portion along the longitudinal direction of the air bag 81 on the back plate 72 side.

The outer edge portion of the air bag 81 is a region around the outer edge of the air bag 81. In the present embodiment, the air bag 81 is configured by welding the two sheet members 86, and thus the weld portions 81a are an example of the outer edge portion of the air bag 81.

As illustrated in FIG. 17, the joining margin 94 is formed by continuously integrating with, for example, the weld portion 91a of the air bag 91 and a portion of the weld portion 92a of the flow path body 92. Thus, a portion of the joining margin 94 is formed on the outer edge portion along the longitudinal direction of the air bag 91. The joining margin 94 is configured, for example, by forming the region of the two sheet members 96 constituting the air bag 91 and the region adjacent to the region constituting the flow path body 92 in the same shape as the air bag 81 and the weld portion 81a on the back plate 72 side of the pressing cuff 71.

As illustrated in FIG. 13, the outer edge portion of the joining margin 94 is formed in the same shape as that of the weld portion 81a of the air bag 81 on the back plate 72 side of the pressing cuff 71. Of the outer edge portions of the joining margin 94, portions facing the weld portions 81a along the longitudinal direction of the air bag 81 constitute joining portions 94a welded to the weld portions 81a. The joining portions 94a are joined to the pressing cuff 71 by welding. In other words, the two outer edge portions of the joining margin 94 along the longitudinal direction of the air bag 91 of the sensing cuff 73 are joined to from one ends to the other ends of the weld portions 81a along the longitudinal direction of the air bag 81. The outer edge portions along the lateral direction of the joining margin 94 are not joined to the weld portions 81a along the lateral direction of the air bag 81.

As a specific example, the sensing cuff 73 having such a configuration includes a fifth sheet member 96a and a sixth sheet member 96b from the wrist 200 side as illustrated in FIGS. 8 and 9. Note that the sensing cuff 73 is constituted by joining adjacent sheet members 96 by welding using heat.

For example, the fifth sheet member 96a and the sixth sheet member 96b are constituted in a shape that can constitute the air bag 91, the flow path body 92, and the joining margin 94. The fifth sheet member 96a and the sixth sheet member 96b are disposed facing one another, are welded using heat along the peripheral edge shape of the air bag 91 and the flow path body 92 so that the air bag 91 and the flow path body 92 are fluidly continuous, and cut in a predetermined shape to constitute the air bag 91, the flow path body 92, and the joining margin 94.

Furthermore, the sixth sheet member 96b includes a hole portion 96b1 into which the leading end of the connection portion 93 can be inserted, for example. The connection portion 93 is disposed in the hole portion 96b1, and the peripheral region of the hole portion 96b1 is welded to the connection portion 93 using heat. The sixth sheet member 96b is joined to the inner circumferential surface of the back plate 72 with the joining layer 75 in between.

As illustrated in FIG. 19, the pressing cuff 71, the back plate 72, and the sensing cuff 73 are integrally constructed by fixing the pressing cuff 71 and the sensing cuff 73 and fixing the back plate 72 to the pressing cuff 71 and the sensing cuff 73, thus constituting the cuff unit 250. The cuff unit 250 is manufactured using a jig 400, which will be described later, having a placement surface 401 configured as a curved surface. Thus, the pressing cuff 71 of the curler 5 is formed in a curved shape following the surface to be joined in a state before being joined to the curler 5.

The tensile cuff 74 is fluidly connected to the pump 14 through the flow path portion 15. As illustrated in FIG. 4, the tensile cuff 74 is inflated to press the curler 5 such that the curler 5 is spaced apart from the wrist 200, pulling the belt 4 and the curler 5 toward the hand back side of the wrist 200. As illustrated in FIGS. 10, 11, 20, and 21, the tensile cuff 74 includes a plurality of, for example, six-layer air bags 101, target join portions 102 provided on the air bag 101 facing the curler 5, the connection portion 103 provided on the air bag 101 facing the curler 5, and notch portions 104 provided in at least the air bag 101 facing the curler 5. The tensile cuff 74 with such a configuration is constituted by welding a plurality of sheet members 106. In addition, the tensile cuff 74 is fixed to the region where the flow path bodies 83 and 92 are provided and the curler 5, including the cover portion 5a, on the hand back side of the wrist 200. In other words, the flow path body 83 of the pressing cuff 71 and the flow path body 92 of the sensing cuff 73 are disposed between the curler 5 on the hand back side of the wrist 200 and the tensile cuff 74.

Additionally, the tensile cuff 74 is configured such that the thickness of the tensile cuff 74 in an inflating direction, in the present embodiment, in the direction in which the curler 5 and the wrist 200 face each other, during inflation, is larger than the thickness of the pressing cuff 71 in the inflating direction during inflation and than the thickness of the sensing cuff 73 in the inflating direction during inflation. Specifically, the air bags 101 of the tensile cuff 74 include more layer structures than the air bags 81 in the pressing cuff 71 and the air bag 91 in the sensing cuff 73, and have thicker thickness than the pressing cuff 71 and the sensing cuff 73 when the air bags 101 are inflated from the curler 5 toward the wrist 200.

As illustrated in FIGS. 10, 11, and 20, in the present embodiment, the tensile cuff 74 including the six-layer air bags 101 includes a first outer layer 111 constituted by one air bag 101, a first intermediate layer 112 constituted by two air bags 101 integrally combining with the first outer layer 111 by welding using heat, a second intermediate layer 113 constituted by two-layer air bags 101 integrally combining with the first intermediate layer 112 by welding using heat, and a second outer layer 114 constituted by one air bag 101 integrally combining with the second intermediate layer 113 by welding using heat.

Here, the air bags 101 are bag-like structures, and in the present embodiment, the blood pressure measuring device 1 is configured to use air with the pump 14, and thus the present embodiment will be described using the air bags. However, in a case where a fluid other than air is used, the bag-like structures may be fluid bags that are inflated by a fluid. A plurality of the air bags 101 are stacked and are in fluid communication in the stacking direction.

Each of the air bags 101 is formed in a rectangular bag-like shape that is long in one direction. Additionally, the air bags 101 are set so that the width in the lateral direction is the same as the width in the lateral direction of the curler 5. The air bag 101 is constituted by, for example, combining two sheet members 106 and, as illustrated in FIGS. 10. 11, 13, 14, 20, and 21, welding weld portions 101a using heat into a rectangular frame shape long in one direction. The six-layer air bags 101 are fluidly continuous through openings provided in the sheet members 106 facing one another.

In addition, in the six-layer air bags 101, for the first outer layer 111 and the first intermediate layer 112, the first intermediate layer 112 and the second intermediate layer 113, and the second intermediate layer 113 and the second outer layer 114, by bridge welding the opposing sheet members 106 together with a quadrilateral frame shape smaller than the weld portion 81a located on the outer peripheral edge and surrounding the plurality of openings with a bridge weld portion 101b, the adjacent air bags 101 are integrally formed and made fluidly continuous on the inner side of the bridge weld portion 101b.

The first outer layer 111 is formed by one air bag 101 disposed on the wrist 200 side. The first outer layer 111 constitutes the first air bag 101 of the six-layer air bags 101 from the wrist 200 side.

The first intermediate layer 112 is stacked on the first outer layer 111. The first intermediate layer 112 is formed by two-layer air bags 101. The first intermediate layer 112 constitutes the second and third air bags 101 of the six-layer air bags 101 from the wrist 200 side. The first intermediate layer 112 is constituted by two-layer air bags 101 integrally welded at the outer peripheral edge. In other words, the first intermediate layer 112 is formed by integrally welding four sheet members 106 in the outer peripheral edge shape of the air bags 101.

The second intermediate layer 113 is stacked on the first intermediate layer 112. The second intermediate layer 113 is formed by two-layer air bags 101. The second intermediate layer 113 constitutes the fourth and fifth air bags 101 of the six-layer air bags 101 from the wrist 200 side. The second intermediate layer 113 is constituted by two-layer air bags 101 integrally welded at the outer peripheral edge. In other words, the second intermediate layer 113 is formed by integrally welding four sheet members 106 in the outer peripheral edge shape of the air bags 101.

The second outer layer 114 is formed by one air bag 101 disposed on the curler 5 side. The second outer layer 114 constitutes the sixth air bag 101 of the six-layer air bags 101 from the wrist 200 side.

One or a plurality of the target join portions 102 are provided on at least a portion of an edge portion of the air bag 101 disposed adjacent to the curler 5. The target join portion 102 is formed by a portion of the sheet member 106 forming the air bag 101.

An example of the present embodiment will be described using examples in which two target join portions 102 are each provided in the longitudinal direction of the air bags 101 on the edge portion in the lateral direction of each of the air bags 101. Note that, for example, the target join portions 102 are provided on the air bags 101 avoiding the positions facing the cover portion 5a of the curler 5. Furthermore, for example, the target join portion 102 includes an escape portion 102a, which is for externally exposing a power feeding terminal 8b described below of the power feeding unit 8 provided on the curler 5, at a portion facing the power feeding terminal 8b. The escape portion 102a, for example, is an opening through which the power feeding terminal 8b can be externally exposed and has a circular shape as an example.

The target join portion 102 is at least joined to the outer circumferential surface of the curler 5 when the tensile cuff 74 is disposed on the inner circumferential surface of the curler 5. Additionally, the target join portions 102 disposed at the same position in the lateral direction of the air bags 101 are stacked and welded.

Note that the two target join portions 102 are set to have a different length to the length in the lateral direction of the air bags 101, for example. In this example, the two target join portions 102 are stacked and welded at the first end side in the lateral direction of the curler 5. Note that as long as the two target join portions 102 are able to be disposed with the leading end on the outer circumferential surface of the curler 5, the length is able to be set as appropriate and the two target join portions 102 may be stackable or not. However, in a case where the length is set to a stackable length, the length is preferably a length such that the leading end does not extend further out than the outer edge of the outer circumferential surface of the curler 5.

The connection portion 103 is, for example, a nipple. The connection portion 103 is provided at a position facing the third hole portion 5f3 of the cover portion 5a in a central region in the longitudinal direction of the air bag 101 disposed adjacent to the curler 5. The leading end of the connection portion 103 is exposed from the sheet member 106 facing the curler 5, of the two sheet members 106 forming the air bag 101. The connection portion 103 is inserted in the third hole portion 5f3 of the cover portion 5a and is connected to the flow path portion 15.

As illustrated in FIG. 19, the notch portion 104 is provided at a position facing the escape portion 5b provided on the curler 5. The notch portion 104 is provided on the sixth air bag 101 forming the second outer layer 114.

In a specific example, as illustrated in FIGS. 10, 11, and 20, the tensile cuff 74 includes a seventh sheet member 106a, an eighth sheet member 106b, a ninth sheet member 106c, a tenth sheet member 106d, an eleventh sheet member 106e, a twelfth sheet member 106f, a thirteenth sheet member 106g, a fourteenth sheet member 106h, a fifteenth sheet member 106i, a sixteenth sheet member 106j, a seventeenth sheet member 106k, and an eighteenth sheet member 106l in this order from the wrist 200 side. Note that the tensile cuff 74 is integrally constituted by joining adjacent sheet members 106 by welding using heat.

The seventh sheet member 106a to the eighteenth sheet member 106l are constituted in a similar rectangular shape to the air bags 101. The seventh sheet member 106a and the eighth sheet member 106b are welded using heat along the peripheral edge portion shape on the four sides of the air bag 101 to constitute the air bag 101 of the first layer from the wrist 200 side. In other words, the seventh sheet member 106a and the eighth sheet member 106b constitute the first outer layer 111.

The eighth sheet member 106b and the ninth sheet member 106c are disposed facing each other, and include a plurality of openings 106b1 and 106c1, respectively, through which the two air bags 101 are fluidly continuous. Additionally, the eighth sheet member 106b and the ninth sheet member 106c are integrally joined by the peripheral region of the plurality of openings 106b1 and 106c1 being bridge welded using heat in a quadrilateral frame shape smaller than the welded four sides of the air bags 101.

The ninth sheet member 106c and the tenth sheet member 106d are welded using heat along the peripheral edge portion shape on the four sides of the air bag 101 to constitute the air bag 101 of the second layer from the wrist 200 side.

As illustrated in FIGS. 10, 11, and 20, the tenth sheet member 106d and the eleventh sheet member 106e are disposed facing each other, and include a plurality of openings 106d1 and 106e1, respectively, through which the two air bags 101 are fluidly continuous. The eleventh sheet member 106e and the twelfth sheet member 106f are welded using heat along the peripheral edge portion shape on the four sides of the air bag 101 to constitute the air bag 101 of the third layer from the wrist 200 side.

The ninth sheet member 106c, the tenth sheet member 106d, the eleventh sheet member 106e, and the twelfth sheet member 106f are integrally welded using heat along the peripheral edge portion shape on the four sides of the air bags 101 to constitute the first intermediate layer 112 in which the second and third air bags 101 are integrally formed.

As illustrated in FIGS. 10, 11, and 20, the twelfth sheet member 106f and the thirteenth sheet member 106g are disposed facing each other, and include a plurality of openings 106f1 and 106g1, respectively, through which the two air bags 101 are fluidly continuous. Additionally, the twelfth sheet member 106f and the thirteenth sheet member 106g are integrally joined by the peripheral region of the plurality of openings 106f1 and 106g1 being bridge welded using heat in a quadrilateral frame shape smaller than the welded four sides of the air bags 101.

The thirteenth sheet member 106g and the fourteenth sheet member 106h are welded using heat along the peripheral edge portion shape on the four sides of the air bag 101 to constitute the air bag 101 of the fourth layer from the wrist 200 side.

As illustrated in FIGS. 10, 11, and 21, the fourteenth sheet member 106h and the fifteenth sheet member 106i are disposed facing each other, and include a plurality of openings 106h1 and 106i1, respectively, through which the two air bags 101 are fluidly continuous. The fifteenth sheet member 106i and the sixteenth sheet member 106j are welded using heat along the peripheral edge portion shape on the four sides of the air bag 101 to constitute the air bag 101 of the fifth layer from the wrist 200 side.

The thirteenth sheet member 106g, the fourteenth sheet member 106h, the fifteenth sheet member 106i, and the sixteenth sheet member 106j are integrally welded using heat along the peripheral edge portion shape on the four sides of the air bags 101 to constitute the second intermediate layer 113 in which the fourth and fifth air bags 101 are integrally formed.

As illustrated in FIGS. 10, 11, and 21, the sixteenth sheet member 106j and the seventeenth sheet member 106k are disposed facing each other, and include a plurality of openings 106j1 and 106k1, respectively, through which the two air bags 101 are fluidly continuous. Also, the seventeenth sheet member 106k, for example, is constituted in a shape that allows the air bag 101 and the target join portion 102 to be constituted. Additionally, the sixteenth sheet member 106j and the seventeenth sheet member 106k are integrally joined by the peripheral region of the plurality of openings 106j1 and 106k1 being bridge welded using heat in a quadrilateral frame shape smaller than the welded four sides of the air bags 101.

The seventeenth sheet member 106k and the eighteenth sheet member 106l are welded using heat along the peripheral edge portion shape on the four sides of the air bag 101 and cut in a predetermined shape to constitute the air bag 101 of the sixth layer from the wrist 200 side, which includes the notch portions 104, and the target join portions 102.

Furthermore, the eighteenth sheet member 106l includes a hole portion 10611 into which the leading end of the connection portion 103 can be inserted, for example. The eighteenth sheet member 106l is disposed with the connection portion 103 at the hole portion 10611, and the peripheral region of the hole portion 10611 is welded to the connection portion 103 using heat. Furthermore, the eighteenth sheet member 106l is joined with the inner circumferential surface of the curler 5 with the joining layer 75 in between, and the target join portion 102 of the seventeenth sheet member 106k is joined to the outer circumferential surface of the curler 5 with the joining layer 75 in between.

Additionally, each of the sheet members 86, 96, and 106 forming the pressing cuff 71, the sensing cuff 73, and the tensile cuff 74 are formed with a thickness of 0.15 mm, for example. Additionally, each of the sheet members 86, 96, and 106 are formed of a thermoplastic resin material. The thermoplastic resin material is a thermoplastic elastomer. Examples of thermoplastic resin material constituting the sheet members 86, 96, and 106 include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin. Note that, in the pressing cuff 71 and the sensing cuff 73, of at least the plurality of sheet members 86 and 106 constituting the air bags 81 and 101, at least the sheet members 86 and 106 welded to the curler 5 are constituted by a material similar to the material of the curler 5.

For example, the sheet members 86, 96, and 106 are formed using a molding method such as T-die extrusion molding or injection molding. After being molded by each molding method, the sheet members 86, 96, and 106 are sized into predetermined shapes, and the sized individual pieces are joined by welding or the like to constitute bag-like structures 81, 91, and 101. A high frequency welder or laser welding is used as the welding method.

The fluid circuit 7 is constituted by the case 11, the pump 14, the flow path portion 15, the on-off valves 16, the pressure sensors 17, the pressing cuff 71, the sensing cuff 73, and the tensile cuff 74. A specific example of the fluid circuit 7 will be described below.

As illustrated in FIG. 5, for example, the fluid circuit 7 includes a first flow path 7a in which the pump 14, the sensing cuff 73, the first pressure sensor 17A and the second pressure sensor 17B are continuous through the first on-off valve 16A, a second flow path 7b which is constituted by branching from the first flow path 7a between the pump 14 and the first on-off valve 16A and is continuous from the pump 14 to the atmosphere through the second on-off valve 16B, the third on-off valve 16C, and the fourth on-off valve 16D sequentially in this order, a third flow path 7c which is constituted by branching from an intermediate portion of the second flow path 7b between the second on-off valve 16B and the third on-off valve 16C and is continuous from the pump 14 to the tensile cuff 74, and a fourth flow path 7d which is constituted by branching from an intermediate portion of the second flow path 7b between the third on-off valve 16C and the fourth on-off valve 16D and is continuous from the pump 14 to the pressing cuff 71.

In the fluid circuit 7 with such a configuration, by the second on-off valve 16B and the third on-off valve 16C being open and the first on-off valve 16A and the fourth on-off valve 16D being closed, the third flow path 7c and the fourth flow path 7d branching from the second flow path 7b are connected to the pump 14, and the pump 14, the pressing cuff 71, and the tensile cuff are fluidly connected.

In the fluid circuit 7, by the first on-off valve 16A, the second on-off valve 16B, and the third on-off valve 16C being open and the fourth on-off valve 16D being closed, the first flow path 7a and the third flow path 7c and the fourth flow path 7d branching from the second flow path 7b are connected to the pump 14, and the pump 14, the pressing cuff 71, and the tensile cuff and the pump 14 and the sensing cuff 73 are fluidly connected. In the fluid circuit 7, by the second on-off valve 16B, the third on-off valve 16C, and the fourth on-off valve 16D being open and the first on-off valve 16A being closed, the second flow path 7b, the third flow path 7c, and the fourth flow path 7d are connected to the pump 14, and the pump 14, the pressing cuff 71, the tensile cuff 74, and the atmosphere are fluidly connected. In the fluid circuit 7, by the first on-off valve 16A, the second on-off valve 16B, the third on-off valve 16C, and the fourth on-off valve 16D being open, the first flow path 7a, the second flow path 7b, the third flow path 7c, and the fourth flow path 7d are connected to the pump 14, and the pump 14, the pressing cuff 71, the sensing cuff 73, the tensile cuff 74, and the atmosphere are fluidly connected.

As illustrated in FIGS. 6 and 7, the power feeding unit 8 is provided in the recess 5c formed in the outer surface of the curler 5 on the first end side that projects from the device body 3. For example, the power feeding unit 8 is configured to be capable to connect to a connector provided on a charging cable of a charger.

As illustrated in FIGS. 3, 6, and 7, the power feeding unit 8 is provided with a wiring portion 8a, the power feeding terminal 8b, and a cover 8c that covers the wiring portion 8a disposed in the recess 5c of the curler 5. The first end of the wiring portion 8a is connected to the power feeding terminal 8b, and the second end is connected to the control unit 55. The power feeding terminal 8b is constituted by two circular terminals, for example. For example, the wiring portion 8a and the power feeding terminal 8b are formed of flexible printed circuits (FPC) and the like including a base film, such as polyimide, provided with an electrically conductive metal film and the like. The cover 8c is formed in the same shape as the recess 5c and covering the recess 5c, and the upper surface runs flush with the outer surface of the curler 5 on the shorter side when the cover 8c is provided in the recess 5c.

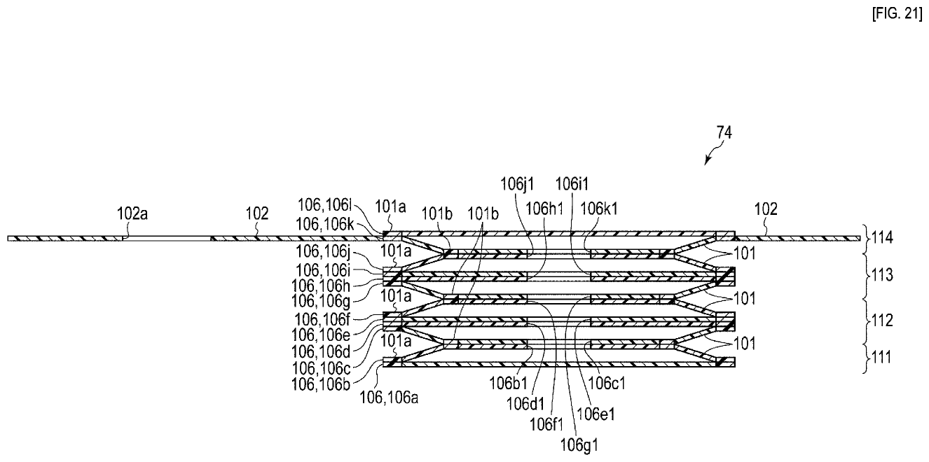

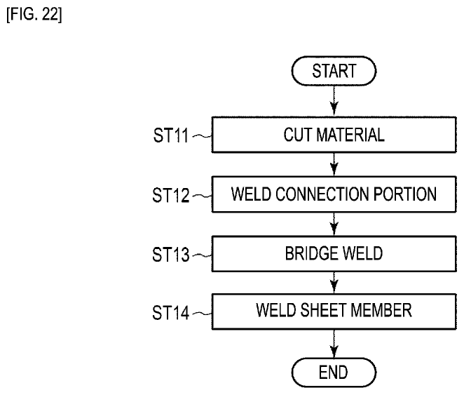

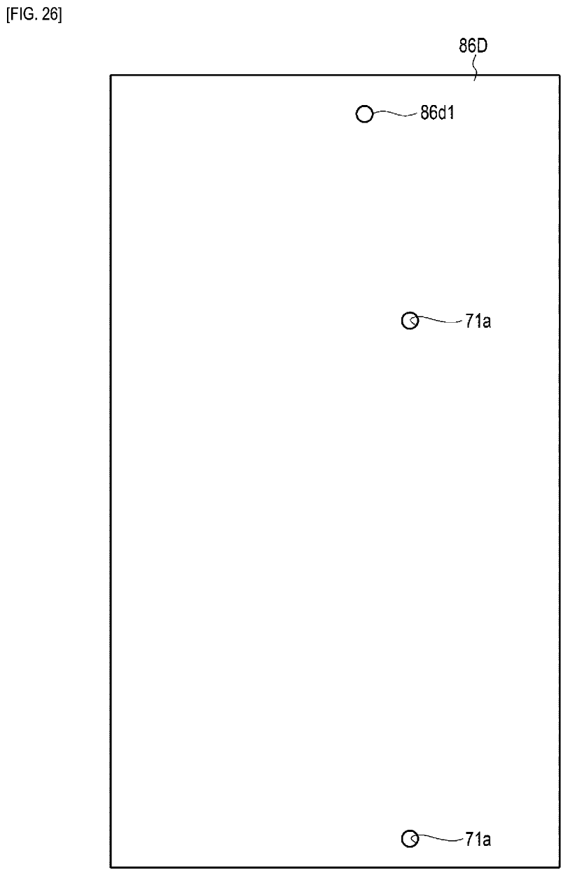

Next, an example of a method for manufacturing a first structure 251 constituting a part of the cuff unit 250 will be described below using FIGS. 22 to 33.

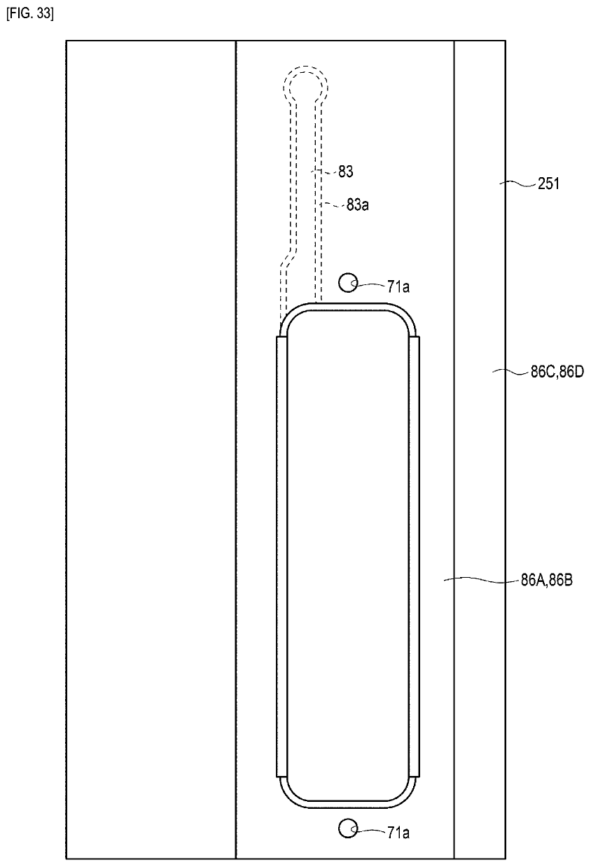

First, as illustrated in FIG. 33, a plurality of sheets constituting the pressing cuff 71 are welded and integrated to constitute the first structure 251.

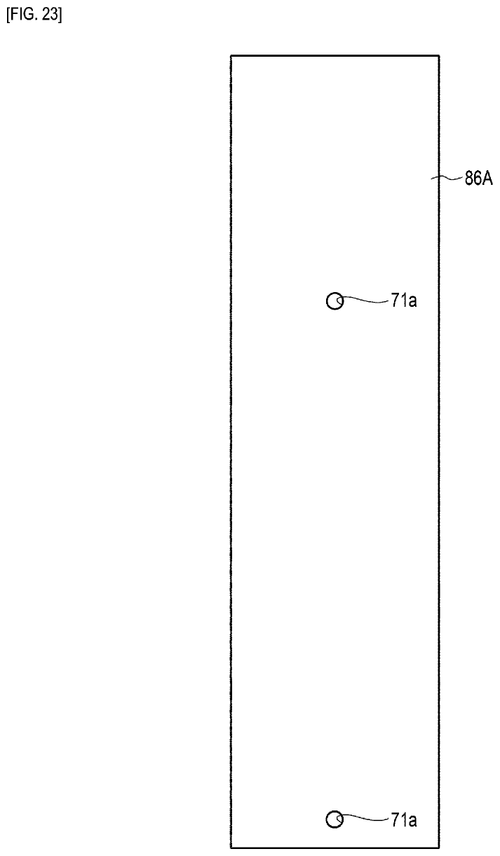

In manufacturing the first structure 251, first, a material is cut (step ST11) to form a sheet in a predetermined shape. Here, the predetermined shape is a shape including the air bag 81 and the target join portion 82, as well as, for example, a welding margin, a dummy portion for alignment in aligning at each of the processing machines. As a specific example, as illustrated in FIGS. 23 to 26, a first sheet 86A, a second sheet 86B, a third sheet 86C, and a fourth sheet 86D are each punched out by a press machine from a sheet-like material formed of a thermoplastic resin material.

As illustrated in FIG. 23, the first sheet 86A includes positioning pin holes 71a into which positioning pins 301 can be inserted, and is a sheet member with a rectangular shape larger than the outer peripheral edge shape of the air bag 81. The first sheet 86A forms the first sheet member 86a. A plurality of, as a specific example, the two pin holes 71a are formed. Each one of the two pin holes 71a is disposed on both sides in the longitudinal direction of the air bag 81 with the region of the first sheet 86A constituting the air bag 81 interspersed therebetween.

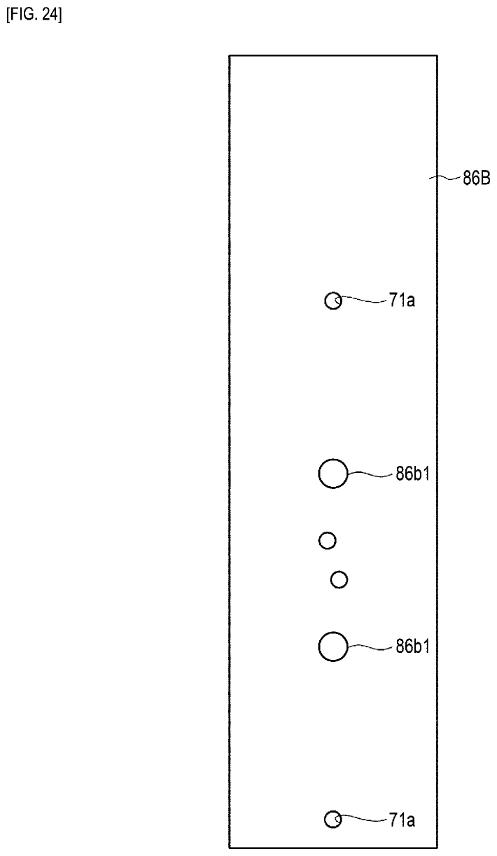

As illustrated in FIG. 24, the second sheet 86B includes the positioning pin holes 71a into which the positioning pins 301 can be inserted and openings through which the adjacent air bag 81 is fluidly communicated, and is a sheet member with a rectangular shape larger than the outer peripheral edge shape of the air bags 81. The second sheet 86B forms the second sheet member 86b. A plurality of, as a specific example, the two pin holes 71a are formed. Each one of the pin holes 71a is disposed on both sides in the longitudinal direction of the air bag 81 with the region of the second sheet 86B constituting the air bag 81 interspersed therebetween. Here, openings in the second sheet 86B constitute openings 86b1 in the second sheet member 86b.

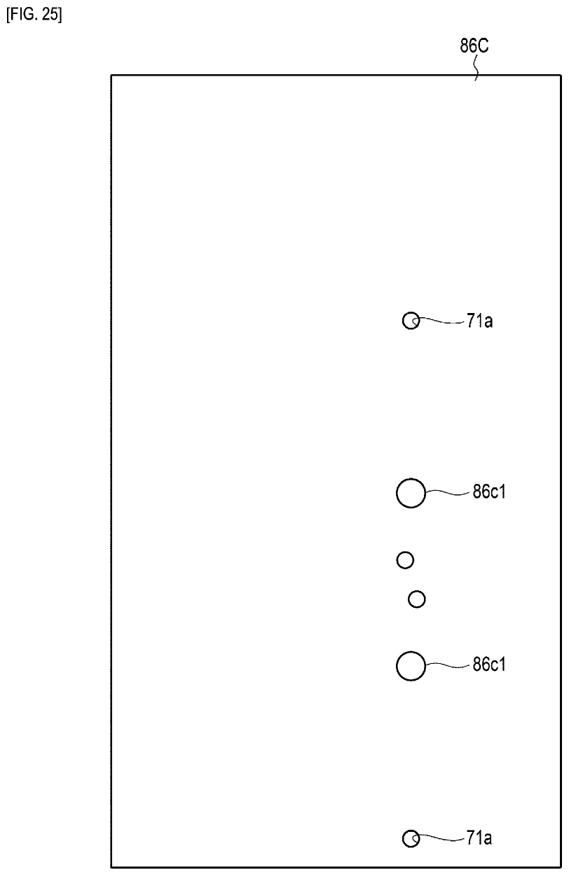

As illustrated in FIG. 25, the third sheet 86C includes the positioning pin holes 71a into which the positioning pins 301 can be inserted and openings through which the adjacent air bag 81 is fluidly connected, and is a sheet member with a rectangular shape larger than the outer peripheral edge shape of the air bag 81 and the target join portion 82. The third sheet 86C forms the third sheet member 86c. A plurality of, as a specific example, the two pin holes 71a are formed. Each one of the pin holes 71a is disposed on both sides in the longitudinal direction of the air bag 81 with the region of the third sheet 86C constituting the air bag 81 interspersed therebetween. Here, openings in the third sheet 86C constitute openings 86c1 in the third sheet member 86c.

The fourth sheet 86D includes the positioning pin holes 71a into which the positioning pins 301 can be inserted and the hole portion 86d1 into which the connection portion 84 is inserted, and is a sheet member with a rectangular shape larger than the outer peripheral edge shape of the air bag 81 and the target join portion 82. The fourth sheet 86D forms the fourth sheet member 86d. A plurality of, as a specific example, the two pin holes 71a are formed. Each one of the pin holes 71a is disposed on both sides in the longitudinal direction of the air bag 81 with the region of the fourth sheet 86D constituting the air bag 81 interspersed therebetween.

Next, as illustrated in FIG. 22, the connection portion 84 is welded to the fourth sheet 86D (step ST12). As a specific example, the connection portion 84 is inserted in the hole portion 86d1 provided in the fourth sheet 86D and the connection portion 84 is welded to the fourth sheet 86D by a high frequency welding machine.

Next, as illustrated in FIGS. 22, 27, and 28, the second sheet 86B and the third sheet 86C are bridge-welded (step ST13). Specifically, the second sheet 86B and the third sheet 86C are layered, the positioning pin holes 71a in the respective second sheet 86B and third sheet 86C are disposed on the positioning pins 301, and the second sheet 86B and the third sheet 86C are disposed in a lower mold 302. As a result, the sheets 86B and 86C are positioned with respect to the lower mold 302. Note that the lower mold 302 is provided with electrode portions 302a, which are projections constituting electrodes. In addition, the electrode portions 302a are formed in a shape abuttable against the region where the second sheet 86B and the third sheet 86C are welded, and an end surface of the electrode portion 302a used in this process is a projection formed in the shape of the bridge weld portion 81b.

Then, bridge-welding is performed in a rectangular frame shape smaller than the outer peripheral edge shapes of the air bags 81 by a high frequency welding machine to form the bridge weld portions 81b, thus integrally welding the second sheet 86B and the third sheet 86C.

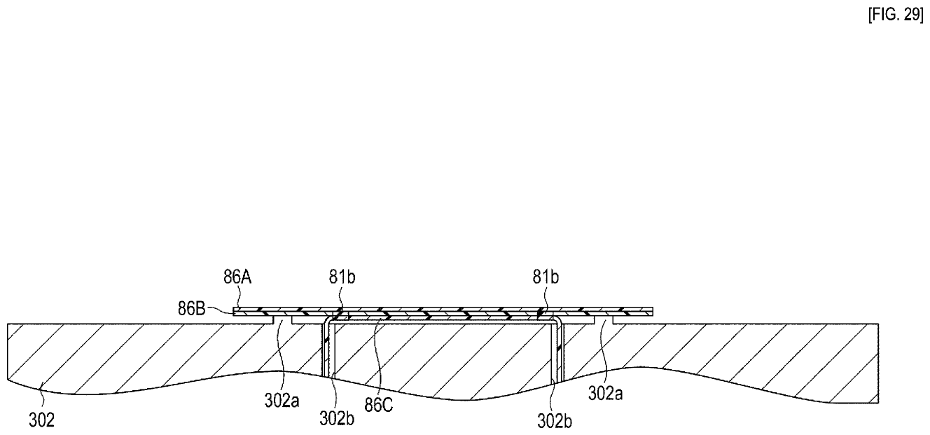

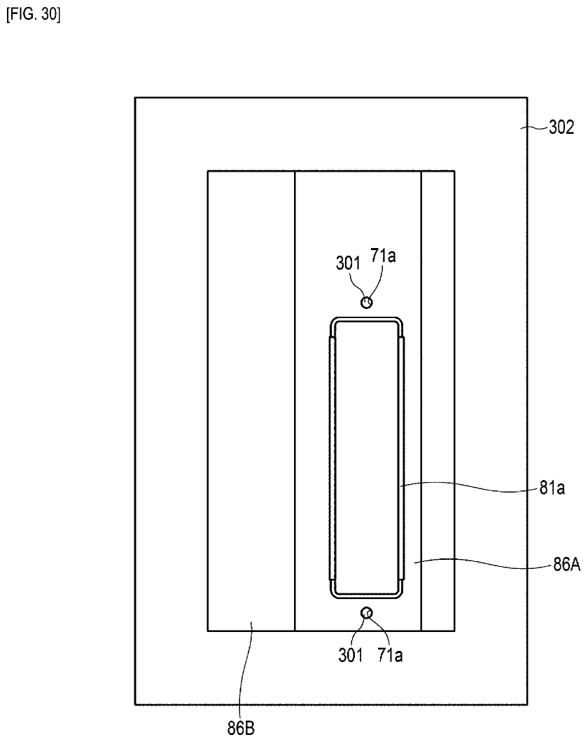

Next, as illustrated in FIGS. 29 and 30, the first sheet 86A is welded to the second sheet 86B. Specifically, the respective positioning pin holes 71a in the second sheet 86B and third sheet 86C, which have been bridge-welded in step ST13, and the first sheet 86A are disposed on the positioning pins 301, and the bridge-welded second sheet 86B and third sheet 86C, and the first sheet 86A are disposed in the lower mold 302. As a result, the sheets 86A, 86B, and 86C are positioned in the lower mold 302. Note that the end surface of the electrode portion 302a of the lower mold 302 used in this process is a projection formed in the shape of the weld portion 81a.

At this time, as illustrated in FIG. 29, at least a portion of the third sheet 86C disposed on the lower mold 302 side and facing the portion where the second sheet 86B and the first sheet 86A are welded is escaped in cavities 302b provided in the lower mold 302. Then, welding is performed on the outer peripheral edge shape of the air bag 81 by a high frequency welding machine to form the weld portion 81a.

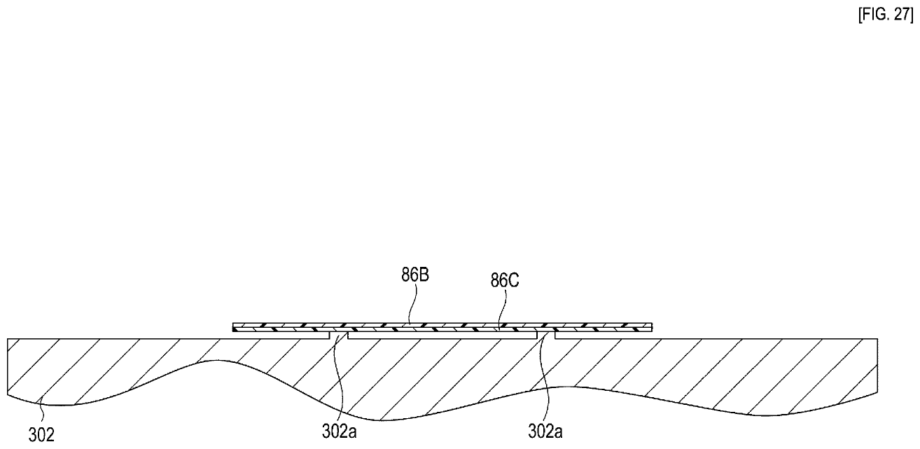

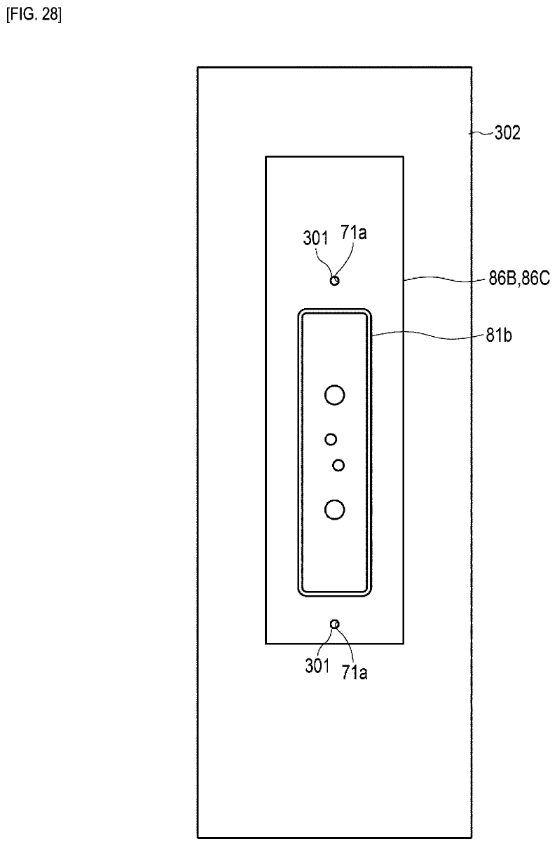

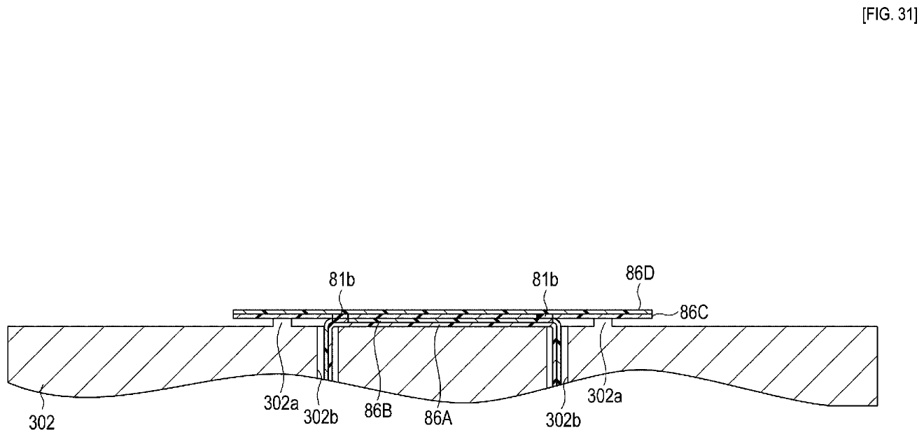

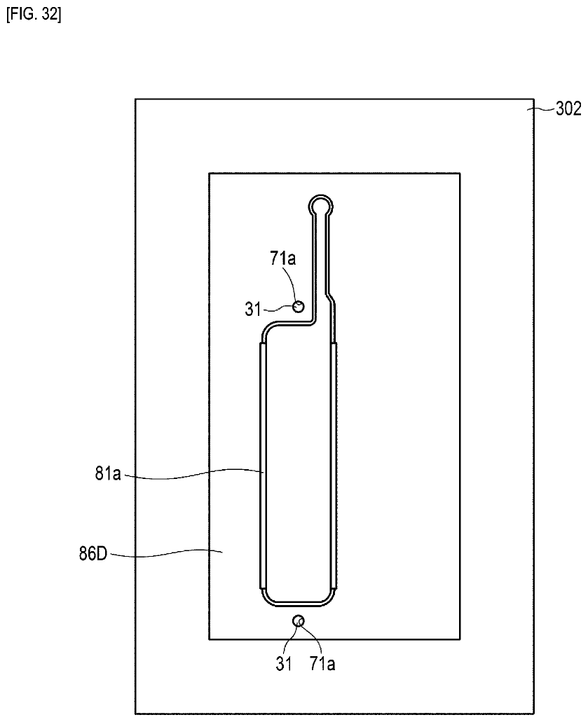

Next, the second sheet 86B, the third sheet 86C, the first sheet 86A, and the fourth sheet 86D that are integrated by welding are welded. Specifically, as illustrated in FIGS. 31 and 32, the respective positioning pin holes 71a in the second sheet 86B, the third sheet 86C, and the first sheet 86A, which are integrated by welding, and the fourth sheet 86D are disposed on the positioning pins 301, and the second sheet 86B, the third sheet 86C, and the first sheet 86A, which are integrated by welding, and the fourth sheet 86D are disposed in the lower mold 302. Here, the second sheet 86B, the third sheet 86C, and the first sheet 86A integrated by welding are the second sheet 86B and the third sheet 86C integrated by bridge welding as illustrated in FIGS. 27 and 28, and the first sheet 86A welded at the weld portions 81a as illustrated in FIGS. 29 and 30. Note that the end surface of the electrode portion 302a of the lower mold 302 used in this process is a projection formed in the shape of the weld portion 81a and the weld portion 83a. In other words, the shape of the end surface of the electrode portion 302a is a shape that can configure the air bag 81 and the flow path body 83.

Also, at this time, as illustrated in FIG. 31, at least a portion of the first sheet 86A and the second sheet 86B facing a portion of the third sheet 86C and the fourth sheet 86D to be welded is escaped in the cavity 302b provided in the lower mold 302. Then, welding is performed on the outer peripheral edge shape of the air bag 81 and the outer peripheral edge shape of the flow path body 83 by a high frequency welding machine to form the weld portion 81a and the weld portion 83a. With these steps, the first structure 251 is manufactured.

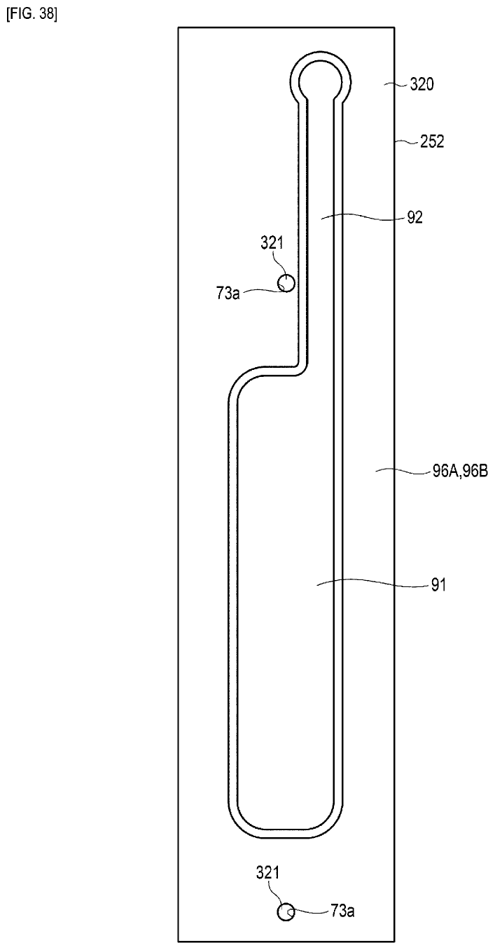

Next, as illustrated in FIG. 38, a plurality of sheets constituting the sensing cuff 73 as a part of the cuff unit 250 are welded and integrated to constitute a second structure 252. Note that, in the present embodiment, an example in which the second structure 252 is manufactured after the first structure 251 is manufactured is described as an example, but the order of manufacturing the first structure 251 and the second structure 252 is not limited. The first structure 251 and the second structure 252 may be concurrently manufactured.

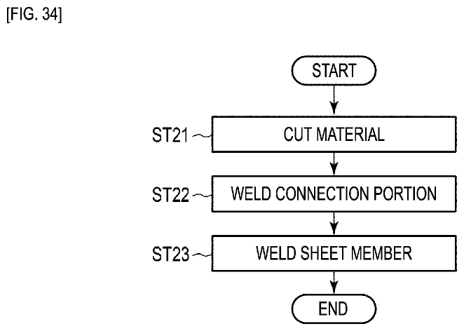

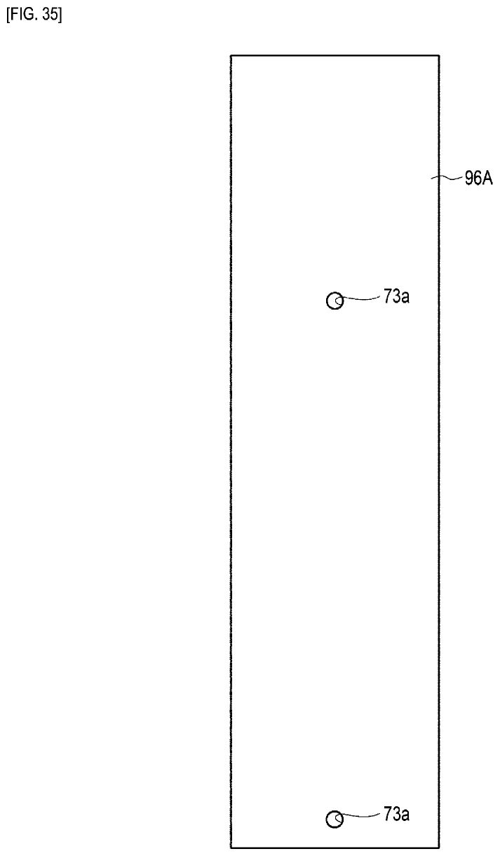

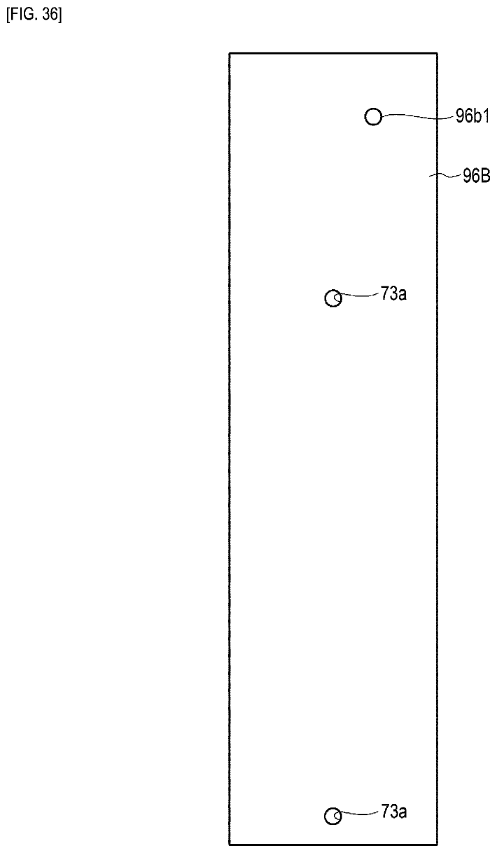

In manufacturing the second structure 252, as illustrated in FIG. 34, first, a material is cut (step ST21) to form a sheet in a predetermined shape. Here, the predetermined shape is a shape including the air bag 91 and the flow path body 92, as well as welding margin, a dummy portion for alignment in aligning at each of the processing machines, and the like. As a specific example, as illustrated in FIGS. 35 and 36, a fifth sheet 96A and a sixth sheet 96B are each punched out by a press machine from a sheet-like material formed of a thermoplastic resin material.

As illustrated in FIG. 35, the fifth sheet 96A has positioning pin holes 73a into which positioning pins 321 can be inserted, and is a sheet member with a rectangular shape larger than the outer peripheral edge shapes of the air bag 91 and the flow path body 92. The fifth sheet 96A forms the fifth sheet member 96a. A plurality of, as a specific example, the two positioning pin holes 73a are formed. Each one of the two pin holes 73a is disposed on both sides in the longitudinal direction of the air bag 91 with the region of the fifth sheet 96A constituting the air bag 91 interspersed therebetween.

As illustrated in FIG. 36, the sixth sheet 96B includes the positioning pin holes 73a into which the positioning pins 321 can be inserted and the hole portion 96b1 into which the connection portion 93 is inserted, and is a sheet member with a rectangular shape larger than the outer peripheral edge shapes of the air bag 91 and the flow path body 92. The sixth sheet 96B forms the sixth sheet member 96b. A plurality of, as a specific example, the two positioning pin holes 73a are formed. Each one of the two pin holes 73a is disposed on both sides in the longitudinal direction of the air bag 91 with the region of the sixth sheet 96B constituting the air bag 91 interspersed therebetween.

Next, as illustrated in FIG. 34, the connection portion 93 is welded to the sixth sheet 96B (step ST22). As a specific example, the connection portion 93 is inserted in the hole portion 96b1 provided in the sixth sheet 96B and the connection portion 93 is welded to the sixth sheet 96B by a high frequency welding machine.

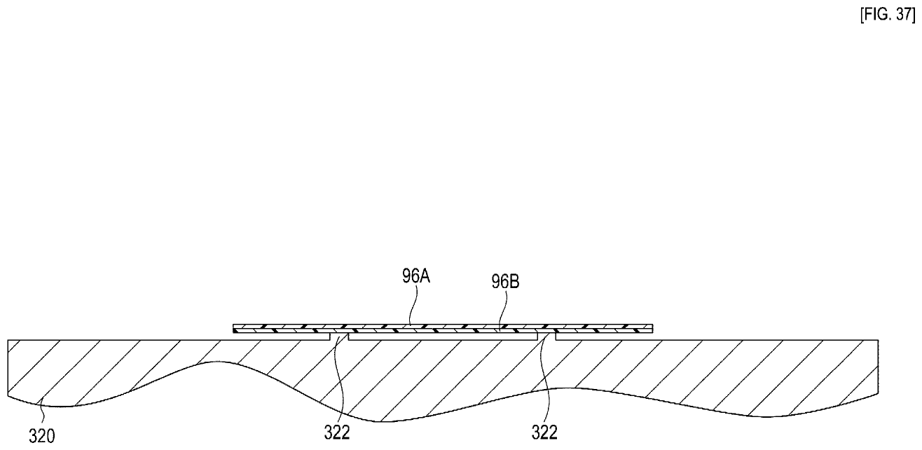

Next, as illustrated in FIGS. 37 and 38, the fifth sheet 96A and the sixth sheet 96B are welded. Specifically, the respective positioning pin holes 73a in the fifth sheet 96A and the sixth sheet 96B are disposed on the positioning pins 321, and the fifth sheet 96A and the sixth sheet 96B are disposed in a lower mold 320. As a result, the fifth sheet 96A and the sixth sheet 96B are positioned with respect to the lower mold 320. Note that the end surface of an electrode portion 322 of the lower mold 320 used in this process is a projection formed in the shape of the weld portion 91a of the air bag 91 and the weld portion 92a of the flow path body 92. Then, welding is performed in the outer peripheral edge shape of the air bag 91 and the outer peripheral shape of the flow path body 92 by a high frequency welding machine to form the weld portions 81a and 91a. With these steps, the second structure 252 is manufactured.

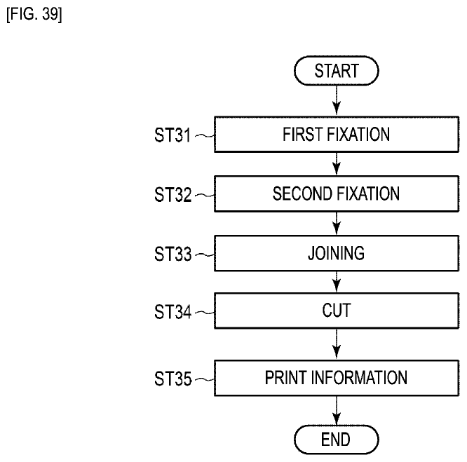

Next, an example of a manufacturing method for manufacturing the cuff unit 250 from the first structure 251, the second structure 252, and the back plate 72 will be described with reference to FIGS. 39 to 43.

First, as illustrated in FIG. 39, a first fixation is performed (step ST31). In the first fixation, the first structure 251 and the back plate 72 are fixed. Specifically, first, the back plate 72 is disposed in a curved jig and a heat treatment is performed by heating in a heating furnace to curve the back plate 72 in a predetermined shape. The predetermined shape here means a shape of the curler 5 along the surface opposed to the back plate 72 via the pressing cuff 71. Next, as illustrated in FIG. 41, the first structure 251 is set in the jig 400.

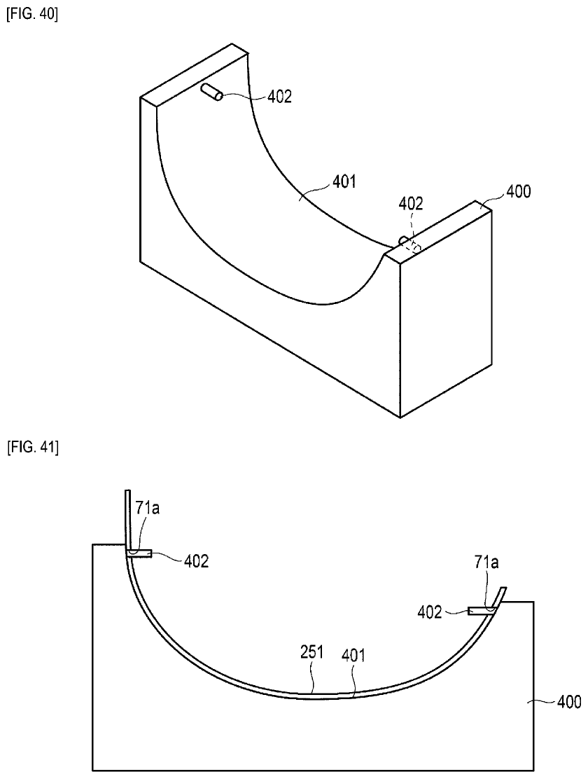

As illustrated in FIG. 40, the jig 400 includes the placement surface 401 and positioning pins 402 for positioning. The placement surface 401 is formed in a curved surface corresponding to the surface of the curler 5 on which the pressing cuff 71 is disposed. The placement surface 401 has a size that is able to dispose at least the entire area of the air bag 81 and the entire area of the air bag 91. Here, “able to be dispose” means that the areas are disposed at positions overlapping with the placement surface 401 in plan view. In other words, this means that the areas can be disposed at the positions where the areas can be curved along the placement surface 401 by direct contact with the placement surface 401, and in the second structure 252, the areas can be disposed at the positions where the areas can be curved along the placement surface 401 by direct contact or indirect contact with the placement surface 401 via at least one of the first structure 251 and the back plate 72.

In the present embodiment, as an example, in a state in which the pins 402 are set in the pin holes 71a and 73a, the placement surface 401 have a size that is able to dispose the entire area of the air bag 81 and a portion of the flow path body 83, and the entire area of the air bag 91 and a portion of the flow path body 92.

Here, the curved surface corresponding to the surface of the curler 5 on which the pressing cuff 71 is disposed is a curved surface that, when the cuff unit 250 formed along this curved surface is fixed to the curler 5, the cuff unit 250 curves more than that before the cuff unit 250 is fixed to the curler 5, and this allows suppressing wrinkles, which inhibit the inflation, in at least one of the pressing cuff 71 and the sensing cuff 73. The placement surface 401 is, for example, a curved surface having a curvature same as that of the surface of the curler 5 on which the pressing cuff 71 is disposed. In another example, the placement surface 401 is a curved surface having a curvature substantially the same as that of the surface of the curler 5 on which the pressing cuff 71 is disposed.

A plurality of, as a specific example, the two positioning pins 402 for positioning are formed. The positioning pins 402 are configured to be able to dispose the respective pin holes 71a in the first structure 251 and the respective pin holes 73a in the second structure 252. The positioning pins 402 for positioning are formed in a shape that can reduce movements of the first structure 251 and the second structure 252 with respect to the jig 400, and as a specific example, are formed in a shape fitting into the pin holes 71a and 73a. The first positioning pin 402 is disposed, for example, on the first end of the placement surface 401. The second pin 402 is disposed, for example, on the second end of the placement surface 401.

The first structure 251 is set to the positioning pins 402 for positioning. As a specific example, the first positioning pin 402 is disposed in the first pin holes 71a in the first structure 251, and the second positioning pin 402 is disposed in the second pin holes 71a. The first pin holes 71a here are one of the pin holes 71a disposed to be opposed to each other among the two pin holes 71a in the respective sheets 86A, 86B, 86C, and 86D. The second pin holes 71a are the second pin holes 71a disposed to be opposed. Next, the first structure 251 placed on the placement surface 401 is pressed against the placement surface 401 to curve the first structure 251 along the placement surface 401.

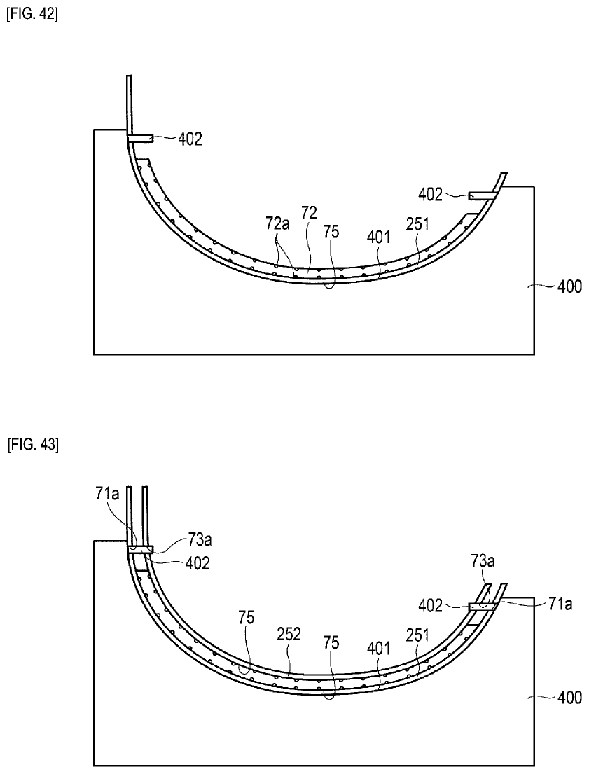

Next, as illustrated in FIG. 42, a double-sided tape is applied to the surface of the air bag 81 opposite to the placement surface 401 of the first structure 251 to form the joining layer 75. Next, the back plate 72 is fixed to the joining layer 75.

Next, a second fixation is performed (step ST32). In the second fixation, the second structure 252 is fixed to the back plate 72. As a specific example, a double-sided tape is applied to the surface of the back plate 72 to form the joining layer 75. Next, as illustrated in FIG. 43, the second structure 252 is set to positioning pins 402 for positioning. As a specific example, the first positioning pin 402 is disposed in the first pin holes 73a in the second structure 252, and the second positioning pin 402 is disposed in the second pin holes 73a. The first pin holes 73a here are one of the pin holes 73a disposed to be opposed to each other among the two pin holes 73a in the respective sheets 96A and 96B. The second pin holes 73a are the second pin holes 73a disposed to be opposed.

Next, the second structure 252 is pressed against the back plate 72 to fix the second structure 252 to the back plate 72.

Next, the joining margin 94 of the first structure 251 is joined to the second structure 252 (step ST33). As a specific example, among the outer edge portions of the joining margin 94, the two edge portions along the longitudinal direction of the air bag 91 and the two edge portions along the longitudinal direction of the weld portions 81a of the air bag 81 on the back plate 72 side of the second structure 252 are welded by a high-frequency welding machine to form the joining portions 94a.

In this way, the first structure 251 and the second structure 252 are integrated by joining the first structure 251 to the second structure 252 at the joining margin 94.

Next, the integrated first structure 251, back plate 72, and second structure 252 are cut to form the shape of the pressing cuff 71 and the shape of the sensing cuff 73 (step ST34). The cut is performed by, for example, a press machine or scissors.

With these steps, the cuff unit 250 is manufactured. Next, information, such as a lot number, is printed on a predetermined location on the manufactured cuff unit 250 (step ST35). The first structure 251 and the second structure 252 are welded on the placement surface 401, which is configured as the curved surface corresponding to the surface of the curler 5 to which the pressing cuff 71 is fixed, and thus the cuff unit 250 configured in this manner is configured to have the shape that curves following the inner circumferential surface of the curler 5 to which the pressing cuff 71 is fixed.

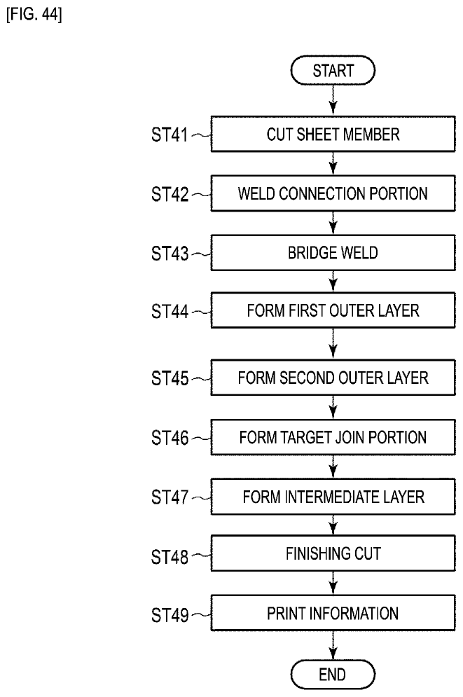

Next, as illustrated in FIG. 44, an example of the method for manufacturing the tensile cuff 74 will be described.

First, a material is cut (step ST41) to form the sheet members 106 in a predetermined shape. Here, the predetermined shape is a shape including the air bags 101 and the target join portion 102, as well as welding margin, a dummy portion for alignment in aligning at each of the processing machines, and the like. As a specific example, the seventh sheet member 106a, the eighth sheet member 106b, the ninth sheet member 106c, the tenth sheet member 106d, the eleventh sheet member 106e, the twelfth sheet member 106f, the thirteenth sheet member 106g, the fourteenth sheet member 106h, the fifteenth sheet member 106i, the sixteenth sheet member 106j, the seventeenth sheet member 106k, and the eighteenth sheet member 106l are each punched out by a press machine from a sheet-like material formed of a thermoplastic resin material.

Next, the connection portion 103 is welded to the eighteenth sheet member 106l (step ST42). As a specific example, the connection portion 103 is inserted in the hole portion 10611, which is provided at the center side of the eighteenth sheet member 106l to weld the connection portion 103 to the eighteenth sheet member 106l by a high frequency welding machine.

Next, bridge welding is performed (step ST43). As a specific example, first, the eighth sheet member 106b and the ninth sheet member 106c are sequentially set to the positioning pins of the jig, and the ninth sheet member 106c is stacked and disposed on the eighth sheet member 106b. Next, the bridge weld portions 101b are formed by welding in a rectangular frame shape by a high frequency welding machine as surrounding around the openings 106b1 and 106c1 of the sheet members 106b and 106c, and the sheet members 106b and 106c are integrally welded.

Next, the twelfth sheet member 106f and the thirteenth sheet member 106g are sequentially set to the positioning pins of the jig, and the thirteenth sheet member 106g is stacked and disposed on the twelfth sheet member 106f. Next, the bridge weld portions 101b are formed by welding in a rectangular frame shape by a high frequency welding machine as surrounding around the openings 106g1 and 106f1 of the sheet members 106g and 106f, and the sheet members 106g and 106f are integrally welded.

Next, the sixteenth sheet member 106j and the seventeenth sheet member 106k are sequentially set to the positioning pins of the jig, and the seventeenth sheet member 106k is stacked and disposed on the sixteenth sheet member 106j. Next, the bridge weld portions 101b are formed by welding in a rectangular frame shape by a high frequency welding machine as surrounding around the openings 106j1 and 106k1 of the sheet members 106j and 106k, and the sheet members 106j and 106k are integrally welded.

Next, the first outer layer 111 is formed (step ST44). Specifically, the eighth sheet member 106b and the ninth sheet member 106c, which have been bridge-welded in step ST43, and the seventh sheet member 106a are sequentially set to the positioning pins of the jig, and welded in the outer peripheral edge shapes of the air bags 101 by a high frequency welding machine to form the weld portions 101a. In this manner, the first outer layer 111 is formed.

Next, the second outer layer 114 is formed (step ST45). Specifically, the sixteenth sheet member 106j and the seventeenth sheet member 106k, which have been bridge-welded in step ST43, and the eighteenth sheet member 106l to which the connection portion 103 has been welded in step ST42 are sequentially set to the positioning pins of the jig, and welded in the outer peripheral edge shapes of the air bags 101 by a high frequency welding machine to form the weld portions 101a. In this manner, the second outer layer 114 is formed.

Next, the target join portions 102 are formed on the second outer layer 114 that has been formed (step ST46). Specifically, the second outer layer 114 is set to the positioning pins of the jig, and the second outer layer 114 formed in step ST45 is disposed in a press mold with a shape that allows the second outer layer 114 to be cut in the outer peripheral edge shapes of the air bag 101 of the sixth layer, the target join portion 102, and the notch portions 104. Next, a contact plate is disposed on the upper surface of the second outer layer 114 and the contact plate is press-processed by a press processing machine. In this manner, the second outer layer 114 is cut in the outer peripheral edge shapes of the air bag 101 of the sixth layer, the target join portions 102, and the notch portions 104, and the target join portions 102 are formed on the second outer layer 114.

Next, the first intermediate layer 112 and the second intermediate layer 113 are formed (step ST47). First, an intermediate electrode is disposed between the twelfth sheet member 106f and the thirteenth sheet member 106g, which have been bridge-welded in step ST43, at the positioning pins of the jig. Next, the second outer layer 114, the sheet members 106h and 106i on which bridge welding has not been performed, the sheet member 106g and 106f with the intermediate electrode disposed therebetween, the sheet members 106d and 106e on which bridge welding has not been performed, and the first outer layer 111 are sequentially layered and each welded in the outer peripheral edge shape of the air bag 101 by a high frequency welding machine to form the weld portion 101a. In this manner, the first intermediate layer 112 and the second intermediate layer 113 are formed. That is, the first outer layer 111, the first intermediate layer 112, the second intermediate layer 113, and the second outer layer 114, i.e. the six-layer air bags 101 are formed.

Next, the first outer layer 111, the first intermediate layer 112, and the second intermediate layer 113 that have been formed are finish-cut (step ST48). With these steps, the tensile cuff 74 is manufactured. Next, information, such as a lot number, is printed on a predetermined location on the manufactured tensile cuff 74 (step ST49).

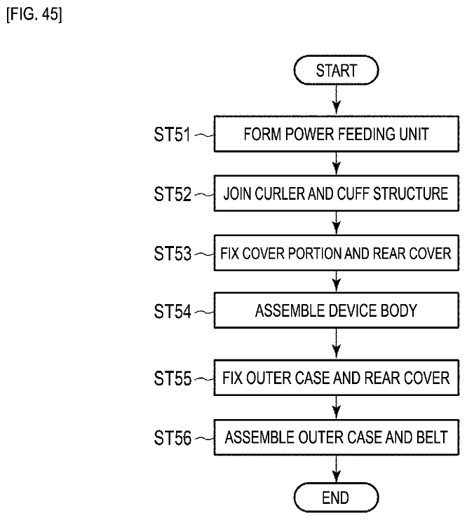

Next, as illustrated in FIG. 45, an example of a method for manufacturing the blood pressure measuring device 1 will be described.

First, the power feeding unit 8 is formed on the curler 5 (step S51). The FPC constituting the wiring portion 8a and the power feeding terminal 8b is joined to the cover portion 5a and the recess 5c of the curler 5 by double-sided tape or the like and the cover 8c is joined to the recess 5c by double-sided tape of the like.

Next, the cuff structure 6 is joined to the curler 5 (step ST52). As a specific example, first, double-sided tapes as the joining layers 75 are attached to a region of the fourth sheet member 86d of the pressing cuff 71 facing the curler 5 of the cuff unit 250 and the target join portion 82, and the pressing cuff 71 is attached to the curler 5. The pressing cuff 71 is fixed to the curler 5 with the joining layer 75, and thus the back plate 72 and the sensing cuff 73 are also attached to the curler 5 at the same time. Note that in this step, the connection portion 84 of the pressing cuff 71 and the connection portion 93 of the sensing cuff 73 are inserted into the first hole portion 5f1 and the second hole portion 5f2 in the cover portion 5a of the curler 5 in advance.

Then, double-sided tape is attached to the region of the eighteenth sheet member 106l of the tensile cuff 74 facing the curler 5 and the target join portion 102, and the tensile cuff 74 is attached to the curler 5 as well as the flow path body 83 of the pressing cuff 71 disposed on the inner surface of the curler 5 and the flow path body 92 of the sensing cuff 73. These steps join the cuff structure 6 to the curler 5.

Next, the sealing member 36 and the rear cover 35 are disposed on the cover portion 5a and the rear cover 35 is fixed to the cover portion 5a with the first joining members 35a (step ST53) to constitute a rear lid.

Then, the device body 3 is integrally assembled except for the rear cover 35 (step ST54). Next, the rear cover 35 is disposed on the end portion on the wrist 200 side of the outer case 31 of the device body 3, and the outer case 31 and the rear cover 35 are fixed with the second joining members 35b (step ST55). Then, the first belt 61 and the second belt 62 are assembled on the outer case 31 (step ST56). With these steps, the blood pressure measuring device 1 is manufactured.

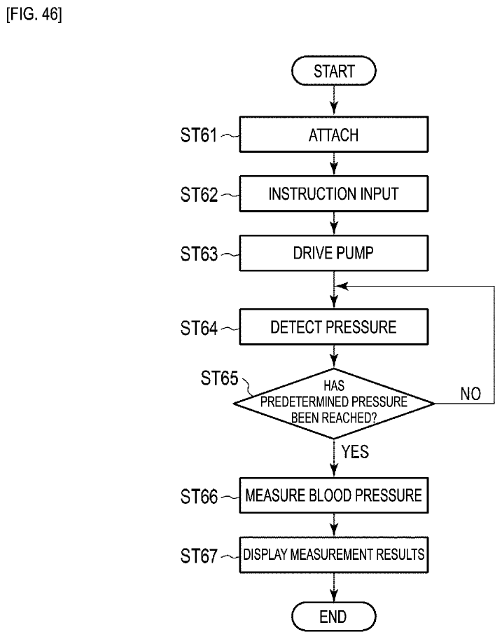

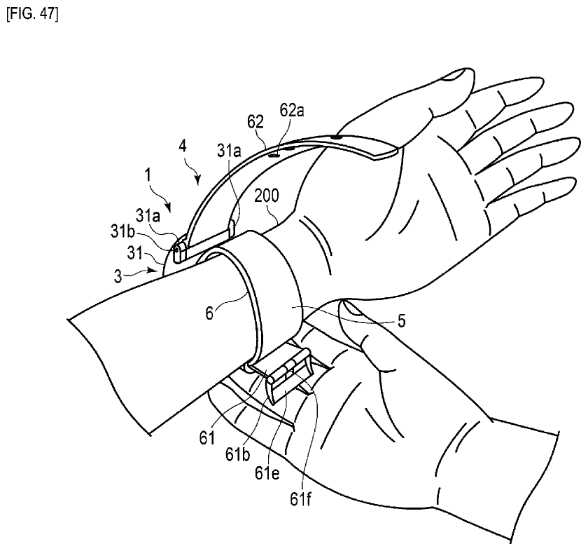

Next, an example of measurement of a blood pressure value using the blood pressure measuring device 1 will be described using FIGS. 46 to 50. FIG. 46 is a flowchart depicting an example of the blood pressure measurement using the blood pressure measuring device 1, illustrating both an operation of the user and an operation of the control unit 55. Additionally, FIGS. 47 to 49 illustrate an example of the user attaching the blood pressure measuring device 1 on the wrist 200.

First, the user attaches the blood pressure measuring device 1 to the wrist 200 (step ST61). As a specific example, for example, the user inserts one of the wrists 200 into the curler 5, as illustrated in FIG. 47.

At this time, in the blood pressure measuring device 1, the device body 3 and the sensing cuff 73 are disposed at opposite positions in the curler 5, and thus the sensing cuff 73 is disposed in a region on the hand palm-side of the wrist 200 in which the artery 210 resides. Thus, the device body 3 and the tensile cuff 74 are disposed on the hand back side of the wrist 200.

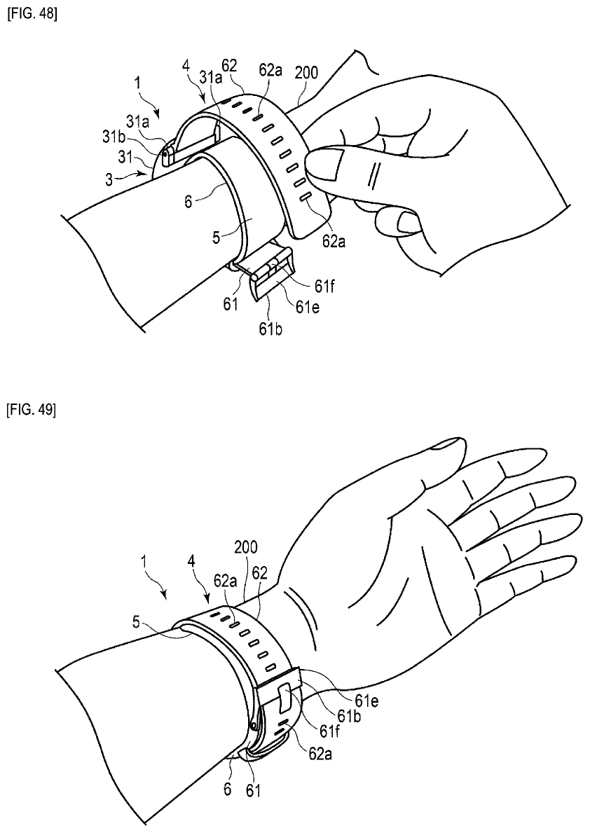

Next, as illustrated in FIG. 48, the user passes the second belt 62 through the frame body 61e of the buckle 61b of the first belt 61 with the hand opposite to the hand on which the blood pressure measuring device 1 is disposed. The user then pulls the second belt 62 to bring the member on the inner circumferential surface side of the curler 5, that is, the cuff structure 6, into close contact with the wrist 200, and inserts the prong 61f into one of the small holes 62a. Thus, as illustrated in FIGS. 4 and 49, the first belt 61 and the second belt 62 are connected, and the blood pressure measuring device 1 is attached to the wrist 200.

Next, the user operates the operation unit 13 and inputs an instruction corresponding to the start of measurement of the blood pressure value. The operation unit 13, on which the input operation of the instruction has been performed, outputs an electrical signal corresponding to the start of the measurement to the control unit 55 (step ST62). The control unit 55 receives the electrical signal, and then, for example, opens the first on-off valve 16A, the second on-off valve 16B, and the third on-off valve 16C, closes the fourth on-off valve 16D, and drives the pump 14 to supply compressed air to the pressing cuff 71, the sensing cuff 73, and the tensile cuff 74 through the first flow path 7a, the second flow path 7b, the third flow path 7c, and the fourth flow path 7d (step ST63). Thus, the pressing cuff 71, the sensing cuff 73, and the tensile cuff 74 start to be inflated.

The first pressure sensor 17A and the second pressure sensor 17B detect the pressures in the pressing cuff 71, the sensing cuff 73, and the tensile cuff 74, and output, to the control unit 55, electrical signals corresponding to the pressures (step ST64). On the basis of the received electrical signals, the control unit 55 determines whether the pressures in the internal spaces of the pressing cuff 71, the sensing cuff 73, and the tensile cuff 74 have reached a predetermined pressure for measurement of the blood pressure (step ST65). For example, in a case where the internal pressures of the pressing cuff 71 and the tensile cuff 74 have not reached the predetermined pressure and the internal pressure of the sensing cuff 73 has reached the predetermined pressure, the control unit 55 closes the first on-off valve 16A and supplies the compressed air through the second flow path 7b, the third flow path 7c, and the fourth flow path 7d.

When the internal pressures of the pressing cuff 71 and the tensile cuff 74 and the internal pressure of the sensing cuff 73 all have reached the predetermined pressure, the control unit 55 stops driving the pump 14 (YES in step ST65). At this time, as illustrated by the two-dot chain line in FIG. 4, the pressing cuff 71 and the tensile cuff 74 are sufficiently inflated, and the inflated pressing cuff 71 presses the back plate 72. Additionally, the tensile cuff 74 presses against the curler 5 in a direction away from the wrist 200, and then the belt 4, the curler 5, and the device body 3 move in a direction away from the wrist 200, and as a result, the pressing cuff 71, the back plate 72, and the sensing cuff 73 are pulled toward the wrist 200 side. In addition, when the belt 4, the curler 5, and the device body 3 move in a direction away from the wrist 200 due to the inflation of the tensile cuff 74, the belt 4 and the curler 5 move toward both lateral sides of the wrist 200, and the belt 4, the curler 5, and the device body 3 move in a state of close contact with both lateral sides of the wrist 200. Thus, the belt 4 and the curler 5, which are in close contact with the skin of the wrist 200, pull the skin on both lateral sides of the wrist 200 toward the hand back side. Note that the curler 5 may be configured to indirectly contact the skin of the wrist 200 with the sheet members 86 or 106 in between, for example, as long as the curler 5 can pull the skin of the wrist 200.

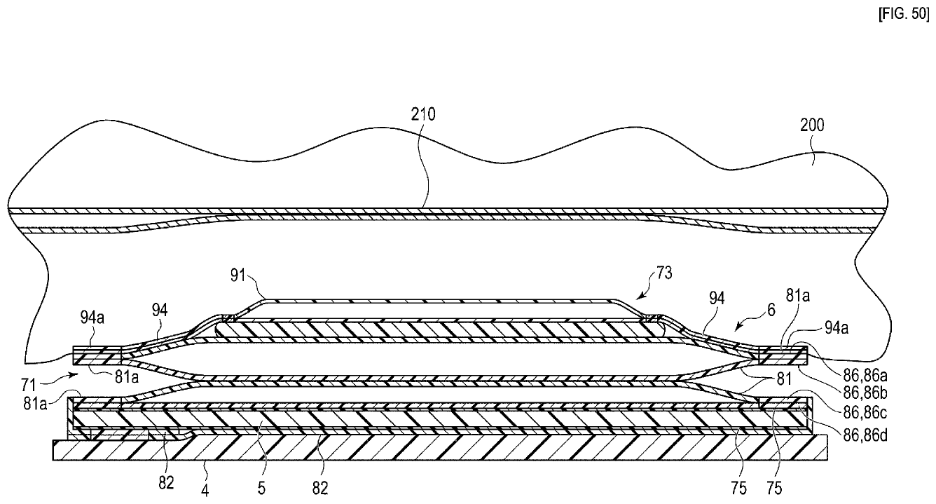

Furthermore, the sensing cuff 73 is inflated by being supplied with a predetermined amount of air such that the internal pressure equals the pressure required to measure blood pressure, and is pressed toward the wrist 200 by the back plate 72 that is pressed by the pressing cuff 71. Thus, the sensing cuff 73 presses the artery 210 in the wrist 200 and occludes the artery 210 as illustrated in FIG. 50.

Additionally, the control unit 55, for example, controls the third on-off valve 16C and repeats the opening and closing of the third on-off valve 16C, or adjusts the degree of opening of the third on-off valve 16C to pressurize a pressure of the internal space of the pressing cuff 71. In the process of pressurization, based on the electrical signal output by the second pressure sensor 17B, the control unit 55 obtains measurement results of, for example, blood pressure values, for example, the systolic blood pressure and the diastolic blood pressure, and the heart rate (step ST66). The control unit 55 outputs image signals corresponding to the obtained measurement results to the display unit 12, and displays the measurement results on the display unit 12 (step ST67). In addition, after the end of the blood pressure measurement, the control unit 55 opens the first on-off valve 16A, the second on-off valve 16B, the third on-off valve 16C, and the fourth on-off valve 16D.

The display unit 12 receives the image signal, and then displays the measurement results on the screen. The user views the display unit 12 to confirm the measurement results. After the measurement is complete, the user removes the prong 61f from the small hole 62a, removes the second belt 62 from the frame body 61e, and pulls out the wrist 200 from the curler 5, thus detaching the blood pressure measuring device 1 from the wrist 200.

In the blood pressure measuring device 1 according to the present embodiment configured in this manner, the joining margin 94 of the sensing cuff 73 is joined to the weld portions 81a of the air bag 81 of the pressing cuff 71 with the joining portions 94a, in other words, are joined to the outer edge portions of the air bag 81. Accordingly, the pressing cuff 71 and the sensing cuff 73 are fixed and integrated, thus constituting the cuff unit 250.

Configuring the integrated cuff unit 250 by fixing the pressing cuff 71 and the sensing cuff 73 in this way allows the pressing cuff 71 and the sensing cuff 73 to be fixed to the curler 5 as the integrated body.

Thus, since the pressing cuff 71 and the sensing cuff 73 can be fixed in a single attachment work to the curler 5, positional displacement of the pressing cuff 71 and the sensing cuff 73 with respect to the curler 5 can be suppressed compared with a configuration in which the pressing cuff 71 and the sensing cuff 73 are separately fixed to the curler 5.

Since the positional deviation of the pressing cuff 71 and the sensing cuff 73 with respect to the curler 5 can be suppressed, it can be suppressed that the position of the sensing cuff 73 with respect to the curler 5 is significantly displaced. As a result, a decrease in the measurement accuracy of the blood pressure measuring device 1 can be suppressed.

Further, configuring the cuff unit 250 eliminates the need for a work to fix the respective pressing cuff 71, back plate 72, and sensing cuff 73 to the curler 5, thereby ensuring improving efficiency of the work to manufacture the blood pressure measuring device 1.

Furthermore, the joining margin 94 is integrally formed with the sensing cuff 73, which makes it possible to suppress the increase in the number of components of the cuff unit 250.

Further, the joining margin 94 is joined to the air bag 81 adjacent to the air bag 91 of the sensing cuff 73 among the plurality of air bags 81 of the pressing cuff 71, and therefore, the inflation of the pressing cuff 71 is not inhibited by the joining margin 94.

Further, the joining margin 94 is formed at the outer edge portion along the longitudinal direction of the air bag 91 of the sensing cuff 73, and the joining portion 94a of the joining margin 94 is joined to the weld portion 81a, which is the outer edge portion along the longitudinal direction of the air bag 81 on the back plate 72 side of the pressing cuff 71. Thus, the joining portion 94a is joined to a wide range of the weld portion 81a of the air bag 81 and a range facing the outside of the curler 5. As a result, this allows suppressing an entrance of water, such as sweat of a user, between the pressing cuff 71 and the sensing cuff 73.

Furthermore, the cuff unit 250 is manufactured with the jig 400. Therefore, in a state prior to the cuff unit 250 being removed from the jig 400 and fixed to the curler 5, the cuff unit 250 is configured to have a shape that curves following the surface of the curler 5 to which the pressing cuff 71 is joined. This allows suppressing a wrinkle, which inhibits inflation, in the pressing cuff 71 and the sensing cuff 73 when the cuff unit 250 is joined to the curler 5.

Furthermore, since the cuff unit 250 includes the back plate 72, the integrated body of the pressing cuff 71, the back plate 72, and the sensing cuff 73 can be fixed to the curler 5 in a single attachment operation, and therefore the number of manufacturing steps of the blood pressure measuring device 1 can be reduced.

Further, by integrally fixing the pressing cuff 71 and the sensing cuff 73 using the jig 400, which has the placement surface 401 configured to have the curved surface corresponding to the surface of the curler 5 to which the pressing cuff 71 is fixed, the pressing cuff 71 and the sensing cuff 73 can be integrally fixed in a curved posture. As a result, when the cuff unit 250 in which the pressing cuff 71 and the sensing cuff 73 are integrally fixed is fixed to the curler 5, a state in which a wrinkle occurs in the air bag 81 of the pressing cuff 71 and the air bag 91 of the sensing cuff 73 and the inflation of the air bag 81 or 91 is inhibited can be suppressed. That is, when the pressing cuff 71 and the sensing cuff 73 are integrally fixed using a jig having a planar placement surface to configure a cuff unit, the cuff unit is configured in which the top surface of the sensing cuff 73 has a planar shape according to the planar placement surface. When this cuff unit is fixed to the curler 5, a difference between the inner circumference and the outer circumference generated by curving the cuff unit according to the shape of the curler 5 possibly generates wrinkles in the air bags 81 and 91.

However, in the present embodiment, using the jig 400 having the placement surface 401, the pressing cuff 71 and the sensing cuff 73 are fixed to be integrated by joining with the joining margin 94. This allows suppressing wrinkles, which inhibit inflation, in the air bags 81 and 91 when the cuff unit 250 is fixed to the curler 5.

Furthermore, the jig 400 has the plurality of pins 402 for positioning, the first structure 251 has the plurality of pin holes 71a, and the second structure 252 has the plurality of pin holes 73a. The first structure 251 and the second structure 252 are fixed to be integrated in a state in which the respective first structure 251 and second structure 252 are set to the pins 402 for positioning. Thus, the positional deviation of the sensing cuff 73 with respect to the pressing cuff 71 can be suppressed.

Furthermore, the pin holes 71a are also used for positioning to the mold 302 with the pins 301 of the mold 302 in the process of manufacturing the first structure 251. Thus, the position of the pressing cuff 71 with respect to the pin holes 71a is fixed. Furthermore, the pin holes 73a are also used for positioning to the mold 320 with the pins 321 of the mold 320 in the process of manufacturing the second structure 252. Thus, the position of the sensing cuff 73 with respect to the pin holes 73a is fixed. In this manner, since the pin holes 71a and 73a are used in the manufacturing process of the structures 251 and 252 and the manufacturing process of the cuff unit 250, it is possible to further suppress the positional deviation of the sensing cuff 73 with respect to the pressing cuff 71.

Furthermore, the plurality of positioning pins 402 are provided, the plurality of respective pin holes 71a and 73a are provided, and the plurality of positioning pins 402 are set in the plurality of pin holes 71a and 73a. As a result, movement of the structures 251 and 252 relative to the placement surface 401, such as rotation around the positioning pins 402, can be suppressed.

Furthermore, after fixing and integrating the first structure 251 and the second structure 252, the cutting is performed, thereby ensuring improving work efficiency of manufacturing the cuff unit 250.

Note that in the blood pressure measuring device 1 of the present embodiment, the configuration in which the joining margin 94 of the sensing cuff 73 is joined at the joining portions 94a to the weld portions 81a, which are provided at the two edge portions along the longitudinal direction of the air bag 81 on the back plate 72 side of the pressing cuff 71, has been described as an example. However, the configuration is not limited thereto. The sensing cuff 73 may be fixed to the pressing cuff 71 at, for example, the annular joining portion 94a surrounding the air bag 81. In this configuration, the joining margin 94 is formed continuously with the weld portion 91a of the air bag 91 and the weld portion 92a of the flow path body 92. The outer edge portion along the longitudinal direction of the joining margin 94 is joined to the weld portion 81a along the longitudinal direction of the air bag 81. The outer edge portion along the lateral direction of the joining margin 94 is joined to the weld portion 81a along the lateral direction of the air bag 81. A portion of the flow path body 92 is fixed to a portion of the weld portion 81a of the air bag 81 on the back plate 72 side of the pressing cuff 71. The flow path body 92 and the weld portion 81a may be fixed by fixing means other than welding. This fixing means is, for example, a double-sided tape or an adhesive.

In the present embodiment, the manufacturing method in which the first structure 251 and the second structure 252 are each manufactured separately and the joining margin 94 of the second structure 252 is joined to the first structure 251 has been described as an example, but the present invention is not limited thereto. As another example, in the manufacturing process of the first structure 251, as described in step ST14 and FIG. 29, in the process of welding the first sheet 86A and the second sheet 86B, which constitute the air bag 81 on the sensing cuff 73 side of the pressing cuff 71, and forming the weld portions 81a, the joining margin 94 of the second structure 252 may be welded and joined at the same time. In other words, the weld portions 81a and the joining portions 94a may be formed by a single welding.

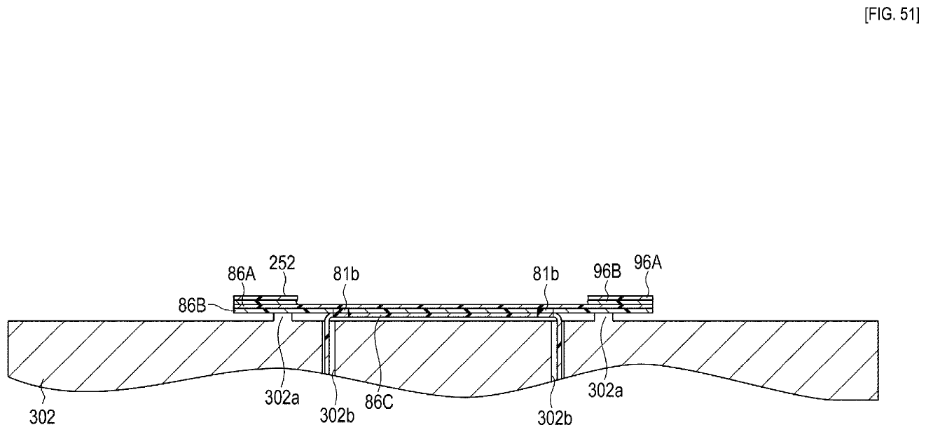

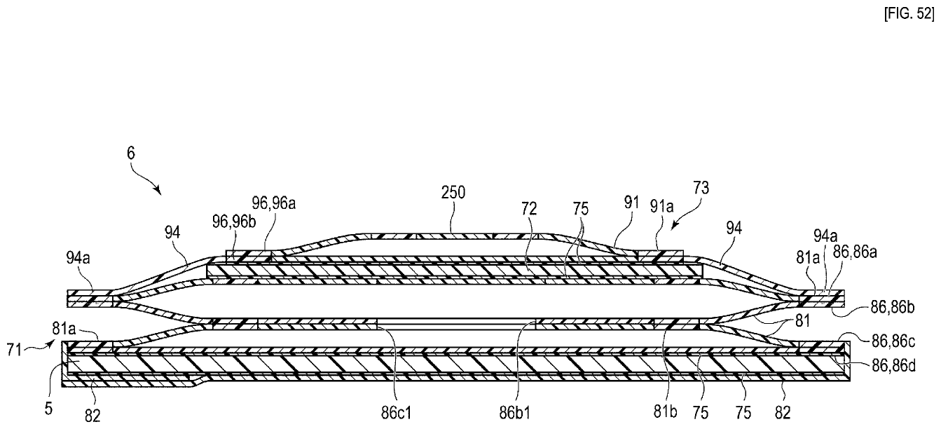

As an example, as illustrated in FIG. 51, the welded second sheet 86B and third sheet 86C are disposed on the mold 302, the first sheet 86A is disposed on the second sheet 86B, and the second structure 252 is further disposed on the first sheet 86A. At this time, the respective pin holes 71a and 73a are disposed on the pins 301 of the mold 302 to be positioned.

Then, by welding the fifth sheet 96A and the sixth sheet 96B constituting the joining margin 94 of the second structure 252 and the first sheet 86A and the second sheet 86B, the weld portions 81a and the joining portions 94a are formed simultaneously. Note that in FIG. 51, only the second structure 252 in the vicinity of the joining portion 94a is illustrated, and other configurations are omitted.

An example of the blood pressure measuring device 1 of the present embodiment in which the joining margin 94 is constituted by the two sheet members 96a and 96b constituting the sensing cuff 73 has been described as an example, but the present invention is not limited thereto. As illustrated in FIGS. 52 and 53, for example, the joining margin 94 may be formed on the sheet member 96a or the sheet member 96b.

As illustrated in FIG. 53, the joining margin 94 is formed on the fifth sheet member 96a disposed on the wrist 200 side. This suppresses a step formed by the thickness of the sheet member 96a on the surface on the wrist 200 side of the sensing cuff 73. Thus, it is possible to suppress the formation of a gap between the wrist 200 and the sensing cuff 73 caused by this step, which makes it possible to suppress a decrease in the measurement accuracy of the blood pressure measuring device 1.

Note that, as illustrated in FIGS. 52 and 53, even in a configuration where the joining margin 94 is formed on the fifth sheet member 96a or the sixth sheet member 96b, as illustrated in FIG. 51, the joining margin 94 and the first sheet 86A and the second sheet 86B, which constitute the air bag 81, may be simultaneously welded to simultaneously form the weld portions 81a and the joining portions 94a. In this case, three sheets are welded simultaneously.

In addition, an example in which the blood pressure measuring device 1 of the present embodiment is manufactured by a manufacturing method that joins the first structure 251 and the second structure 252 with the joining portions 94a of the joining margin 94 to be integrated and then cutting is performed to form the shape of the pressing cuff 71 and to form the shape of the sensing cuff 73 has been described, but the method is not limited to this.

As another example, after forming the first structure 251, the first structure 251 may be cut to form a shape close to the pressing cuff 71, and after the second structure 252 is formed, the second structure 252 may be cut to form a shape close to the sensing cuff 73, and these may be joined with the joining portions 94a using the jig 400 to integrally fix them. Note that the shape close to the pressing cuff 71 here means a shape that leaves a cutting margin to the extent that the pin holes 71a are configured in the outer shape of the pressing cuff 71. The shape close to the sensing cuff 73 means a shape that leaves a cutting margin to the extent that the pin holes 73a are configured in the outer shape of the sensing cuff 73. Then, after the pressing cuff 71 and the sensing cuff 73 are integrated, the cutting margin that constitutes the pin holes 71a and the cutting margin that constitutes the pin holes 73a are cut.

Additionally, in the blood pressure measuring device 1 of the present embodiment, as illustrated in FIGS. 29 to 31, in the welding step (step ST14) of the sheet members of the first structure 251, a step in which the first sheet 86A is welded and integrated to the second sheet 86B of the integrated second sheet 86B and third sheet 86C that have been bridge-welded, and the fourth sheet 86D is welded to the first sheet 86A, the second sheet 86B, and the third sheet 86C, which have been welded and integrated, has been described as an example, but the present invention is not limited thereto. The welding step (step ST14) of the sheet members of the first structure 251 may perform, for example, welding of the second sheet 86B of the second sheet 86B and the third sheet 86C that have been bridge-welded and integrated to the first sheet 86A and welding of the third sheet 86C of the second sheet 86B and the third sheet 86C that have been bridge-welded and integrated to the fourth sheet 86D in one step. An example of this includes the following method.

First, an intermediate electrode is disposed between the bridge-welded second sheet 86B and third sheet 86C. Then, the first sheet 86A, the second sheet 86B and the third sheet 86C in which the intermediate electrode is disposed, and the fourth sheet 86D to which the connection portion 84 is welded are sequentially set to the positioning pins of the jig, and the sheets 86A, 86B, 86C, and 86D are stacked and disposed. Then, the weld portions 81a and 83a are formed by the sheets 86A, 86B, 86C, and 86D being welded in the outer peripheral shape of the pressing cuff 71 by a high frequency welding machine, and the sheets 86A, 86B, 86C, and 86D are integrally welded. In this manner, the air bags 81 and the flow path body 83 are formed.

In the blood pressure measuring device 1 of the present embodiment, as the configuration in which the joining margin 94 is joined to the outer edge portion of the air bag 81 of the pressing cuff 71, the configuration of being joined to the weld portion 81a has been described as an example, but the configuration is not limited thereto. The joining margin 94 may be fixed to a region inside the outer edge and around the outer edge in the outer surface of the air bag 81. Here, the region inside the outer edge and around the outer edge in the outer surface of the air bag 81 is one example of the outer edge portion of the air bag 81.

Note that the present invention is not limited to the embodiments described above. In the embodiment described above, the configuration in which thermal welding is used as joining means for joining the joining margin 94 of the sensing cuff 73 to the pressing cuff 71 has been described as an example, but the present invention is not limited thereto. As another example, welding other than heat may be used. As other examples, the joining means may be an adhesive or a double-sided tape.

In addition, in the present embodiment, the manufacturing method in which after the back plate 72 is disposed on the first structure 251 and fixed with the joining layer 75, the second structure 252 is disposed on the first structure 251 and the back plate 72, and the second structure and the back plate 72 are fixed with the joining layer 75, and after that the first structure 251 and the second structure 252 are fixed and integrated by welding has been described as an example, but the present invention is not limited thereto.

For example, after the second structure 252 is disposed on the first structure 251 and the first structure 251 and the second structure 252 are fixed and integrated by welding, the back plate 72 may be disposed between the first structure 251 and the second structure 252 to fix the back plate 72 to the first structure 251 and the second structure 252.

In the present embodiment, the manufacturing method in which the first structure 251, the back plate 72, and the second structure 252 are sequentially disposed on the placement surface 401 of the jig 400 has been described as an example, but the present invention is not limited thereto. As another example, after integrally fixing the first structure 251 and the back plate 72, the integral body may be set to the positioning pins 402 of the jig 400, subsequently, the second structure 252 may be set to the positioning pins 402, and then the back plate 72 and the second structure 252 may be fixed, and then the first structure 251 and the second structure 252 may be fixed and integrated by welding.

In addition, in the present embodiment, an example in which the first structure 251 and the second structure 252 are each formed into the flat plate shape, and then placed on the placement surface 401 of the jig 400, and thus the first structure 251 and the second structure 252 are curved along the placement surface 401 has been described as an example, but the present invention is not limited thereto. The first structure 251 and the second structure 252 each may be formed with a mold having a placement surface configured as a curved surface corresponding to the surface of the curler 5 on which the pressing cuff 71 is disposed to form the weld portions 81a, 83a, 91a, and 92a in a state of being curved along the curved surface corresponding to the surface of the curler 5 on which the pressing cuff 71 is disposed.

In the present embodiment, an example of the configuration in which the back plate 72 is fixed to the pressing cuff 71 and the sensing cuff 73 with the joining layers 75 formed from the double-sided tape has been described as an example, but the present invention is not limited thereto. The back plate 72 may be fixed by fixing means other than the double-sided tape, such as an adhesive.

In addition, in the present embodiment, the configuration in which the placement surface 401 of the jig 400 has the size that is able to dispose the entire region of the air bag 81, the entire region of the air bag 91, a portion of the flow path body 83, and a portion of the flow path body 92 as the configuration in which at least the entire region of the air bag 81 and the air bag 91 are able to be disposed has been described as an example, but the present invention is not limited thereto. The placement surface 401 may have a size that is able to dispose the entire area of the air bag 81, the entire area of the flow path body 83, the entire area of the air bag 91, and the entire area of the flow path body 92.

In the present embodiment, as the configuration in which the two air bags 81 are provided in the pressing cuff 71 has been described as an example of the configuration in which the plurality of air bags 81 are provided, but the present invention is not limited thereto. The pressing cuff 71 may include the three or more air bags 81, for example.

That is, the present invention is not limited to the embodiments described above, and various modifications can be made in an implementation stage within a range that does not depart from the gist of the present invention. Furthermore, each of the embodiments may be implemented in combination as appropriate to the extent possible, and in this case, combined effects can be obtained. Also, the embodiments described above include various stages of invention, and various inventions may be extracted by appropriately combining the described plurality of disclosed constituent elements.

### REFERENCE SIGNS LIST

1 Blood pressure measuring device
3 Device body
4 Belt
5 Curler
5a Cover portion
5b Escape portion
5c Recess
5d Insert member
5e Screw hole
5f Hole portion
5f1 First hole portion
5f2 Second hole portion
5f3 Third hole portion
6 Cuff structure
7 Fluid circuit
7a First flow path
7b Second flow path
7c Third flow path
7d Fourth flow path
8 Power feeding unit
8a Wiring portion
8b Power feeding terminal
8c Cover
11 Case
12 Display unit
13 Operation unit
14 Pump
15 Flow path portion
16 On-off valve
16A First on-off valve
16B Second on-off valve
16C Third on-off valve
16D Fourth on-off valve
17 Pressure sensor
17A First pressure sensor
17B Second pressure sensor
18 Power supply unit
19 Vibration motor
20 Control substrate
31 Outer case
31a Lug
31b Spring rod
32 Windshield
33 Base
35 Rear cover
35a First joining member
35b Second joining member
35c Hole portion
35d Hole portion
36 Sealing member
41 Button
42 Sensor
43 Touch panel
51 Substrate
52 Acceleration sensor
53 Communication unit
54 Storage unit
55 Control unit
56 Main CPU
57 Sub-CPU
61 First belt
61a Belt portion
61b Buckle
61c First hole portion
61d Second hole portion
61e Frame body
61f Prong
62 Second belt
62a Small hole
62b Third hole portion
71 Pressing cuff
71a Pin hole
72 Back plate
72a Groove
73 Sensing cuff
73a Pin hole
74 Tensile cuff
75 Joining layer
81 Air bag (first bag-like structure)
81a Weld portion
81b Bridge weld portion
82 Target join portion
83 Flow path body
83a Weld portion
84 Connection portion
86 Sheet member
86a First sheet member
86b Second sheet member
86b1 Opening
86c Third sheet member
86c1 Opening
86d Fourth sheet member
86d1 Hole portion
91 Air bag (second bag-like structure)
91a Weld portion
92 Flow path body
92a Weld portion
93 Connection portion
94 Joining margin
94a Joining portion
96 Sheet member
96a Fifth sheet member
96b Sixth sheet member
96b1 Hole portion
101 Air bag
101a Weld portion
101b Bridge weld portion
102 Target join portion
102a Escape portion
103 Connection portion
104 Notch portion
106 Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106g Thirteenth sheet member
106g1 Opening
106h Fourteenth sheet member
106h1 Opening
106i Fifteenth sheet member
106i1 Opening
106j Sixteenth sheet member
106j1 Opening
106k Seventeenth sheet member
106k Sheet member
106k1 Opening
1061 Eighteenth sheet member
10611 Hole portion
111 First outer layer
112 First intermediate layer
113 Second intermediate layer
114 Second outer layer
200 Wrist
250 Cuff unit
251 First structure
252 Second structure
210 Artery
301 Positioning pin
302 Lower mold
302a Electrode portion
302b Cavity
320 Lower mold
321 Positioning pin
322 Electrode portion
400 Jig
401 Placement surface
402 Positioning pin

1. A cuff unit comprising:
a pressing cuff that includes a plurality of first bag-like structures, the plurality of first bag-like structures being layered and each inflated by a fluid, the pressing cuff being joined to a curler; and
a sensing cuff that includes one second bag-like structure and a joining margin, the one second bag-like structure being inflated by a fluid, the joining margin being formed on the second bag-like structure and joined to the first bag-like structure adjacent to the second bag-like structure.

2. The cuff unit according to claim 1, wherein
the first bag-like structure is configured in a shape long in one direction;
the second bag-like structure is configured in a shape long in one direction; and
the joining margin is formed at an outer edge portion along a longitudinal direction of the second bag-like structure, and the joining margin is joined to the outer edge portion along the longitudinal direction of the first bag-like structure.

3. The cuff unit according to claim 1, wherein
the pressing cuff and the sensing cuff are curved following an inner circumferential surface of the curler.

4. The cuff unit according to claim 1, comprising
a back plate disposed between the pressing cuff and the sensing cuff and joined to the pressing cuff and the sensing cuff.

5. A method for manufacturing a cuff unit comprising:
disposing a first structure including a pressing cuff on a placement surface of a jig to position the first structure with respect to the placement surface formed in a curved surface corresponding to a surface of a curler to which the pressing cuff is joined;
disposing a second structure including a sensing cuff on the first structure to position the second structure with respect to the placement surface; and
joining and integrating the first structure and the second structure.

6. The method for manufacturing the cuff unit according to claim 5, wherein
the second structure includes a joining margin; and
the first structure and the second structure are joined at the joining margin.

7. The method for manufacturing the cuff unit according to claim 5, comprising
after disposing a back plate on the first structure and joining the back plate to the first structure, disposing the second structure on the first structure and the back plate to join the second structure to the first structure and the back plate.

8. The method for manufacturing the cuff unit according to claim 6, wherein
the jig includes a plurality of positioning pins;
the first structure includes a plurality of holes in which the positioning pins are disposed;
the second structure includes a plurality of holes in which the positioning pins are disposed; and
the joining margin is joined to the first structure with the plurality of positioning pins set to the plurality of holes in the first structure and the plurality of holes in the second structure.

9. The method for manufacturing the cuff unit according to claim 5, wherein
the first structure and the second structure each include a cutting margin; and
after the first structure and the second structure are fixed and integrated, the cutting margins are cut.

10. A blood pressure measuring device comprising:
a curler;
a cuff unit that includes a pressing cuff and a sensing cuff, the pressing cuff including a plurality of first bag-like structures, the plurality of first bag-like structures being layered and each inflated by a fluid, the pressing cuff being joined to a curler, the sensing cuff including one second bag-like structure and a joining margin, the one second bag-like structure being inflated by a fluid, the joining margin being formed on the second bag-like structure and joined to the first bag-like structure adjacent to the second bag-like structure; and
a device body that supplies the fluid to the cuff unit.
